(19) United States
(12) Patent Application Publication
(10) Pub. No.: US 2003/0004113 A1
(43) Pub. Date: Jan. 2, 2003

(54) Methods for modulating an immune response by modulating the interaction between CTLA4 and PP2A

(76) Inventors: Joaquin Madrenas, London (CA); Mary Collins, Natick, MA (US); Beatriz Carreno, Acton, MA (US)

Correspondence Address: LAHIVE & COCKFIELD, 28 STATE STREET, BOSTON, MA, 02109, US

(21) Appl. No.: 10/211,207

(22) Filed: Aug. 2, 2002

Related U.S. Application Data
(63) Continuation of application No. 10/077,106, filed on Feb. 15, 2002.
(60) Provisional application No. 60/269,757, filed on Feb. 16, 2001.

(51) Int. Cl. A61K 38/10; A61K 38/08

(52) U.S. Cl. 514/15

(57) ABSTRACT

The present invention provides methods for modulating an immune response comprising contacting a cell with an agent that modulates the interaction between CTLA4 and PP2AA via modulating the lysine rich motif of CTLA4. The invention further provides methods for treating a subject having a disorder that would benefit from down regulation of an immune response comprising administering an agent that modulates the interaction between CTLA4 and PP2AA via modulating the lysine rich motif of CTLA4. The invention also provides methods for identifying compounds capable of modulating the interaction of CTLA4 and PP2AA.

### RELATED APPLICATIONS

This application claims priority to U.S. Ser. No. 60/269,757, filed on Feb. 16, 2001. The entire contents of that application are incorporated herein by reference.

### BACKGROUND OF THE INVENTION

In order for T cells to respond to foreign polypeptides, two signals must be provided by antigen-presenting cells (APCs) to resting T lymphocytes (Jenkins, M. and Schwartz, R. (1987) J. Exp. Med. 165:302-319; Mueller, D. L. et al. (1990) J. Immunol. 144:3701-3709). The first signal, which confers specificity to the immune response, is transduced via the T cell receptor (TCR) following recognition of foreign antigenic peptide presented in the context of the major histocompatibility complex (MHC). The second signal, termed costimulation, induces T cells to proliferate and become functional (Lenschow et al. (1996) Annu. Rev. Immunol. 14:233). Costimulation is neither antigen-specific, nor MHC-restricted, and is thought to be provided by one or more distinct cell surface molecules expressed by APCs (Jenkins, M. K. et al. (1988) J. Immunol. 140:3324-3330; Linsley, P. S. et al. (1991) J. Exp. Med. 173:721-730; Gimmi, C. D. et al. (1991) Proc. Natl. Acad. Sci. USA 88:6575-6579; Young, J. W. et al. (1992) J. Clin. Invest. 90:229-237; Koulova, L. et al. (1991) J. Exp. Med. 173:759-762; Reiser, H. et al. (1992) Proc. Natl. Acad. Sci. USA 89:271-275; van-Seventer, G. A. et al. (1990) J. Immunol. 144:4579-4586; LaSalle, J. M. et al. (1991) J. Immunol. 147:774-80; Dustin, M. I. et al. (1989) J. Exp. Med. 169:503; Armitage, R. J. et al. (1992) Nature 357:80-82; Liu, Y. et al. (1992) J. Exp. Med. 175:437-445).

The CD80 (B7-1) and CD86 (B7-2) proteins, expressed on APCs, are critical costimulatory molecules (Freeman et al. (1991) J. Exp. Med. 174:625; Freeman et al. (1989) J. Immunol. 143:2714; Azuma et al. (1993) Nature 366:76; Freeman et al. (1993). Science 262:909). B7-2 appears to play a predominant role during primary immune responses, while B7-1, which is upregulated later in the course of an immune response, may be important in prolonging primary T cell responses or costimulating secondary T cell responses (Bluestone (1995) Immunity 2:555).

One ligand to which B7-1 and B7-2 bind, CD28, is constitutively expressed on resting T cells and increases in expression after activation. After signaling through the T cell receptor, ligation of CD28 and transduction of a costimulatory signal induces T cells to proliferate and secrete IL-2 (Linsley, P. S. et al. (1991) J. Exp. Med. 173:721-730; Gimmi, C. D. et al. (1991) Proc. Natl. Acad. Sci. USA 88:6575-6579; June, C. H. et al. (1990) Immunol. Today 11:211-6; Harding, F. A. et al. (1992) Nature 356:607-609). A second ligand, termed CTLA4 (CD152) is homologous to CD28 but is not expressed on resting T cells and appears following T cell activation (Brunet, J. F. et al. (1987) Nature 328:267-270). CTLA4 appears to be critical in negative regulation of T cell responses (Waterhouse et al. (1995) Science 270:985). Blockade of CTLA4 has been found to remove inhibitory signals, while aggregation of CTLA4 has been found to provide inhibitory signals that downregulate T cell responses (Allison and Krummel (1995) Science 270:932). The B7 molecules have a higher affinity for CTLA4 than for CD28 (Linsley, P. S. et al. (1991) J. Exp. Med. 174:561-569) and B7-1 and B7-2 have been found to bind to distinct regions of the CTLA4 molecule and have different kinetics of binding to CTLA4 (Linsley et al. (1994) Immunity 1:793). A new molecule related to CD28 and CTLA4, ICOS, has been identified (Hutloff et al. (1999) Nature 397:263; WO 98/38216), as has its ligand, which is a new B7 family member (Aicher A. et al. (2000) J. Immunol. 164:4689-96; Mages H. W. et al. (2000) Eur. J. Immunol. 30:1040-7; Brodie D. et al. (2000) Curr. Biol. 10:333-6; Ling V. et al. (2000) J. Immunol. 164:1653-7; Yoshinaga S. K. et al. (1999) Nature 402:827-32).

Immune cells have receptors that transmit activating signals. For example, T cells have T cell receptors and the CD3 complex, B cells have B cell receptors, and myeloid cells have Fc receptors. In addition, immune cells bear receptors that transmit signals that provide costimulatory signals or receptors that transmit signals that inhibit receptor-mediated signaling. For example, CD28 transmits a costimulatory signal to T cells. After ligation of the T cell receptor, ligation of CD28 results in a costimulatory signal characterized by, e.g., upregulation of IL-2rα, IL-2rβ, and IL-2rγ receptor, increased transcription of IL-2 messenger RNA, and increased expression of cytokine genes (including IL-2, IFN-γ, GM-CSF, and TNF-α). Transmission of a costimulatory signal allows the cell to progress through the cell cycle and, thus, increases T cell proliferation (Greenfield et al. (1998) Critical Reviews in Immunology 18:389). Binding of a receptor on a T cell which transmits a costimulatory signal to the cell (e.g., ligation of a costimulatory receptor that leads to cytokine secretion and/or proliferation of the T cell) by a costimulatory ligand results in costimulation. Thus, inhibition of an interaction between a costimulatory ligand and a receptor that transmits a costimulatory signal on immune cells results in a downmodulation of the immune response and/or specific unresponsiveness, termed immune cell anergy. Inhibition of this interaction can be accomplished using, e.g., anti-CD28 Fab fragments, antibodies to B7 family molecules, or by using a soluble form of a receptor to which a B7 family member molecule can bind as a competitive inhibitor (e.g., CTLA4Ig).

Inhibitory receptors that bind to costimulatory molecules have also been identified on immune cells. Activation of CTLA4, for example, transmits a negative signal to a T cell (Carreno et al.(2000) J. Immunol. 165:1352. Engagement of CTLA4 inhibits IL-2 production and can induce cell cycle arrest (Krummel and Allison (1996) J. Exp. Med. 183:2533). In addition, mice that lack CTLA4 develop lymphoproliferative disease (Tivol et al. (1995) Immunity 3:541; Waterhouse et al. (1995) Science 270:985). The blockade of CTLA4 with antibodies may remove an inhibitory signal, whereas aggregation of CTLA4 with antibody transmits an inhibitory signal. Therefore, depending upon the receptor to which a costimulatory molecule binds (i.e., a costimulatory receptor such as CD28 or an inhibitory receptor such as CTLA4), B7 molecules can promote T cell costimulation or inhibition.

The importance of the B7:CD28/CTLA4 costimulatory pathway has been demonstrated in vitro and in several in vivo model systems. Blockade of this costimulatory pathway results in the development of antigen-specific tolerance in murine and human systems (Harding, F. A. et al. (1992) Nature 356:607-609; Lenschow, D. J. et al. (1992) Science 257:789-792; Turka, L. A. et al. (1992) Proc. Natl. Acad. Sci. USA 89:11102-11105; Gimmi, C. D. et al. (1993) Proc. Natl. Acad. Sci. USA 90:6586-6590; Boussiotis, V. et al. (1993) J. Exp. Med. 178:1753-1763). Conversely, expression of B7 by B7-negative murine tumor cells induces T-cell mediated specific immunity accompanied by tumor rejection and long lasting protection to tumor challenge (Chen, L. et al. (1992) Cell 71:1093-1102; Townsend, S. E. and Allison, J. P. (1993) Science 259:368-370; Baskar, S. et al. (1993) Proc. Natl. Acad. Sci 90:5687-5690.). Therefore, manipulation of the costimulatory pathways offers great potential to stimulate or suppress immune responses in humans.

Activation of T lymphocytes through their antigen receptor (TCR) induces upregulation of CTLA4 expression (Chambers, C. A. and Allison, J. P. (1999) Curr. Opin. Cell Biol. 11:203-210; Slavik, J. M. et al. (1999) Immunol. Res. 19:1-24 (1999); Oosterwegel, M. A. et al. (1999) Curr. Opin. Immunol. 11:294-300; Ravetch, J. V. and Lanier, L. L. (2000) Science 290:84-89; Sansom, D. M. (2000) Immunology 101:169-177). Subsequent coligation of CTLA4 with the TCR inhibits T cell responses by at least two mechanisms—antagonism of CD28 costimulation by sequestration of B7 and delivery of a negative signal into T cells (Baroja et al. 2000. J. Immunol. 164:49; Carreno, B. M. et al. (2000) J. Immunol. 65:1352-1356; Masteller, E. L. et al. (2000) J. Immunol. 164:5319-5327). The inhibitory function of CTLA4 has made it a potentially important therapeutic target for the treatment of cancer, autoimmune diseases, and transplant rejection. Although blockade of CTLA4 has been easy to achieve and is currently in early stages of clinical development (Leach, D. R. et al. (1996) Science 271:1734-1736; Abrams, J. R. et al. (1999) J. Clin. Invest. 103:1243-1252), enhancement of CTLA4 function has not been possible because of the lack of sufficient knowledge about the mechanism by which CTLA4 inhibits T cell activation and function. Furthermore, nothing is known about the regulation of CTLA4 function, for example, within the CTLA4 molecule, it is not clear which interactions and regions of the molecule need to be targeted to enhance its inhibitory function. Such an enhancement of its function would be of direct value in turning off unwanted immune responses.

### SUMMARY OF THE INVENTION

The present invention is based, at least in part, on the discovery that the regulatory subunit of the serine/threonine phosphatase 2A (PP2AA) interacts with the cytoplasmic tail of CTLA4; that T cell receptor (TCR) ligation induces tyrosine phosphorylation of PP2AA and its dissociation from CTLA4 when coligated; that the association between PP2AA and CTLA4 involves a conserved three-lysine motif in the cytoplasmic tail of CTLA4; and that mutation of these lysine residues in the lysine-rich motif prevents the binding of PP2AA and enhances the inhibition of IL-2 gene transcription by CTLA4. These discoveries indicate that interaction of PP2A with CTLA4 represses CTLA4 function, and thus promotes immune responses.

Accordingly, one embodiment of the present invention provides a method for modulating an immune response comprising contacting a cell expressing at least one first molecule having a CTLA4 lysine rich motif and at least one second molecule having a PP2AA CTLA4-interacting domain with an agent that modulates the interaction between the first molecule and the second molecule to thereby modulate the immune responses. The method may be performed either in vitro or in vivo. In a preferred embodiment, the cell is a T cell. In a further embodiment, anergy is induced in the T cell.

In one embodiment, the agent interacts with the lysine rich motif of CTLA4. In another embodiment, the agent interacts with amino acid residues 392-589 of PP2AA. In another embodiment, the agent is selected from the group consisting of: a peptide comprising the amino acid sequence SKMLKKRSP (SEQ ID NO:1), a peptide that binds to a PP2AA molecule, a peptide that binds to a CTLA4 molecule, a CTLA4 cytoplasmic domain or a portion thereof, a peptide comprising residues 392-589 of PP2AA, and a small molecule.

In a preferred embodiment, the interaction between the first molecule and the second molecule is downregulated. In another preferred embodiment, the immune response is downregulated. In a further embodiment, the cell is contacted with at least one additional agent that downregulates an immune response.

Another embodiment of the invention provides a method for treating a subject having a condition that would benefit from downregulation of an immune response comprising administering an agent that inhibits the interaction between interaction between a first molecule having a having a CTLA4 lysine rich motif and a second molecule having a PP2AA CTLA4-interacting domain in at least T cell of the subject such that a condition that would benefit from downregulation of an immune response is treated.

In one embodiment, the interaction between the first molecule and the second molecule is downregulated. In another embodiment, signaling via a T cell receptor in at least one T cell of the subject is downregulated. In yet another embodiment, anergy is induced in at least one T cell of the subject. In a further embodiment, the method comprises administering to the subject at least one additional agent that downregulates an immune response.

In one embodiment, the agent interacts with the lysine rich motif of CTLA4. In another embodiment, the agent interacts with amino acid residues 392-589 of PP2AA. In still another embodiment, the agent is selected from the group consisting of: a peptide comprising the amino acid sequence SKMLKKRSP (SEQ ID NO:1), a peptide that binds to a PP2AA, a peptide that binds to a CTLA4 molecule, a CTLA4 cytoplasmic domain or a portion thereof, a peptide comprising residues 392-589 of PP2AA, and a small molecule.

In one embodiment, the condition is an autoimmune disorder (e.g., rheumatoid arthritis, myasthenia gravis, autoimmune thyroiditis, systemic lupus erythematosus, type I diabetes mellitus, Grave's disease, and multiple sclerosis). In another embodiment, the condition is a transplant (e.g., a bone marrow transplant, a stem cell transplant, a heart transplant, a lung transplant, a liver transplant, a kidney transplant, a cornea transplant, or a skin transplant). In another embodiment, the condition is graft versus host disease. In yet another embodiment, the condition is an allergy. In yet another embodiment, the condition is an inflammatory disorder.

Another embodiment of the invention provides a method for treating a subject having a condition that would benefit from downregulation of an immune response, comprising contacting T cells expressing at least one first molecule having a having a CTLA4 lysine rich motif and at least one second molecule having a PP2AA CTLA4-interacting domain from the subject with an agent that modulates the interaction between the first molecule and the second molecule, and administering the T cells to the subject, such that a condition that would benefit from downregulation of an immune response is treated.

In one embodiment, the interaction between the first molecule and the second molecule is downregulated. In another embodiment, signaling via T cell receptors in the T cells from the subject is downregulated. In still another embodiment, anergy is induced in the T cells of the subject. In a further embodiment, the method comprises administering to the subject at least one additional agent that downregulates an immune response.

In one embodiment, the agent interacts with the lysine rich motif of CTLA4. In another embodiment, the agent interacts with amino acid residues 392-589 of PP2AA. In yet another embodiment, the agent is selected from the group consisting of: a peptide comprising the amino acid sequence SKMLKKRSP (SEQ ID NO:1), a peptide that binds to a PP2AA molecule, a peptide that binds to a CTLA4 molecule, a CTLA4 cytoplasmic domain or a portion thereof, a peptide comprising residues 392-589 of PP2AA, and a small molecule.

In one embodiment the condition is an autoimmune disorder (e.g., rheumatoid arthritis, myasthenia gravis, autoimmune thyroiditis, systemic lupus erythematosus, type I diabetes mellitus, Grave's disease, and multiple sclerosis). In another embodiment, the condition is a transplant (e.g., a bone marrow transplant, a stem cell transplant, a heart transplant, a lung transplant, a liver transplant, a kidney transplant, a cornea transplant, or a skin transplant). In another embodiment, the condition is graft versus host disease. In yet another embodiment, the condition is an allergy. In still another embodiment, the condition is an inflammatory disorder.

Another embodiment of the invention provides a method for identifying a compound which modulates the interaction of CTLA4 and PP2AA comprising contacting a cell comprising at least one first molecule (e.g., an exogenous first molecule) having a CTLA4 cytoplasmic domain containing a CTLA4 lysine rich motif and at least one second molecule (e.g., an exogenous second molecule) having a PP2AA CTLA4-interacting domain with a test compound and determining the ability of the test compound to modulate the interaction of the first molecule and second molecule.

In one embodiment, the second molecule comprises amino acid residues 392-589 of PP2AA.

In a preferred embodiment, the interaction of the first molecule and the second molecule is inhibited.

In one embodiment, determining the ability of the test compound to modulate the interaction of the first molecule and the second molecule comprises determining the ability of the test compound to modulate the coimmunoprecipitation of the first molecule and the second molecule.

In one preferred embodiment, the cell is a yeast cell. In a further embodiment, determining the ability of the test compound to modulate the interaction of the first molecule and the second molecule comprises determining the ability of the compound to modulate growth of the yeast cell on nutritionally selective media. In another embodiment, determining the ability of the test compound to modulate the interaction of the first molecule and the second molecule comprises determining the ability of the compound to modulate expression of a LacZ reporter gene in the yeast cell.

In another preferred embodiment, the cell is a T cell. In a further embodiment, determining the ability of the test compound to modulate the interaction of the first molecule and the second molecule comprises determining the ability of the test compound to modulate cytokine production by the T cell. In another embodiment, determining the ability of the test compound to modulate cytokine production by the T cell comprises determining the ability of the compound to modulate the activity of a reporter gene operatively linked to the IL-2 promoter/enhancer region in the T cell. In still another embodiment, determining the ability of the test compound to modulate the interaction of the first molecule and the second molecule comprises determining the ability of the test compound to modulate proliferation of the T cell.

Another embodiment of the invention provides a method for identifying a compound which modulates the interaction of a CTLA4 molecule and a PP2AA molecule comprising contacting, in the presence of the compound, a first molecule comprising at least a portion of the CTLA4 molecule and a second molecule comprising at least a portion of the PP2AA molecule under conditions which allow binding of the first molecule and the second molecule to form a complex; and detecting the formation of a complex of the first molecule and the second molecule in which the ability of the compound to modulate interaction between the first molecule and the second molecule is indicated by a change in complex formation as compared to the amount of complex formed in the absence of the compound. In a preferred embodiment, the formation of a complex of the first molecule and the second molecule is inhibited by the compound.

In a preferred embodiment, the first molecule comprises a CTLA4 cytoplasmic domain. In another preferred embodiment, the first molecule comprises at least one lysine rich motif. In another embodiment, the second molecule comprises amino acid residues 392-589 of PP2AA.

In one embodiment, detecting the formation of a complex of the first molecule and the second molecule comprises detecting coimmunoprecipitation of the first molecule and the second molecule.

Another embodiment of the invention provides a method for identifying a compound which modulates the interaction of a molecule comprising at least one CTLA4 lysine rich motif and a PP2AA molecule comprising a PP2AA CTLA4-interacting domain comprising contacting the molecule comprising at least one CTLA4 lysine rich motif with the compound and detecting binding of the compound to the lysine rich motif of the molecule, to thereby identify a compound which modulates the interaction of a molecule comprising at least one CTLA4 lysine rich motif and a PP2AA molecule. In a further embodiment, the molecule comprising at least one CTLA4 lysine rich motif consists of at least one CTLA4 lysine rich motif.

### BRIEF DESCRIPTION OF THE DRAWINGS

FIG. 1 depicts an alignment of lysine containing regions from a number of known PP2A binding proteins. Bold residues are those conserved in different molecules. The sequences are arranged from more to less conservation of the three main residues of the lysine rich motif (as defined herein).

FIGS. 2A-2B depict the inhibition of IL-2 promoter/enhancer controlled luciferase reporter gene transcription by wild type (FIG. 2A) or K-less (FIG. 2B) CTLA4 molecules. Wild type or K-less CTLA4-transfected T cells were stimulated for 4 hours with antigen presenting cells and increasing concentrations of SEE antigen in the absence (upper data points) or presence (lower data points) of doxycycline (5 μg/ml). Cells were lysed and a luciferase assay was performed.

### DETAILED DESCRIPTION OF THE INVENTION

The present invention is based, at least in part, on the discovery that the regulatory subunit of the serine/threonine phosphatase 2A (also referred to herein as PP2AA) interacts with the cytoplasmic tail of CTLA4; that T cell receptor (TCR) ligation induces tyrosine phosphorylation of PP2AA and its dissociation from CTLA4 when coligated; that the association between PP2AA and CTLA4 involves a conserved three-lysine motif in the cytoplasmic tail of CTLA4; and that mutation of these lysine residues in the lysine-rich motif prevents the binding of PP2AA and enhances the inhibition of IL-2 gene transcription by CTLA4, indicating that PP2A represses CTLA4 function.

PP2A is a multimeric eukaryotic serine/threonine phosphatase involved in a wide range of cellular processes. Based on the discoveries of the instant invention, modulation of the interaction between PP2AA and CTLA4 is a way to modulate CTLA4 activity without affecting PP2A activity with respect to other cellular processes. Broad inhibition of PP2A activity is known to be toxic. For example, the PP2A inhibitor okadaic acid, which accumulates in filter feeding organisms such as shellfish, causes diarrhetic shellfish poisoning when consumed by humans (Schönthal, A. H. (1998) Front. Biosci. 3:1262-1273; Murakami, Y. et al. (1982) Bull. Jap. Soc. Sci. Fish. 48:69-72; Tachibana, K. et al. (1981) J. Am. Chem. Soc. 103:2469-2471).

Accordingly, the present invention provides methods for modulating immune responses and treating immune disorders by targeting the interaction between CTLA4 and PP2AA. The present invention further provides methods for identifying compounds which can modulate the interaction between PP2AA and CTLA4, e.g., by binding the lysine rich motif of CTLA4.

Various aspects of the invention are described in further detail in the following subsections:

I. Definitions

As used interchangeably herein, the terms “PP2A” and “PP2A holoenzyme” refers to a protein phosphatase which is found in all eukaryotic cells and which has a wide range of biological functions, including regulation of the cell cycle, signal transduction, cytoskeleton organization, immediate early gene transcription, protein biosynthesis, and cholesterol biosynthesis. PP2A enzymatically removes phosphate groups from proteins and is typically found as heterotrimeric enzyme made up of a catalytic subunit, also referred to interchangeably herein as a “C subunit”, “PP2A subunit C”, or “PP2AC”, and two regulatory subunits, a “B subunit”, also referred to interchangeably herein as a “PP2A regulatory subunit B” or “PP2AB”, and an “A subunit”, also referred to interchangeably herein as “PP2A regulatory subunit A”, or “PP2AA” (Oliver, C. J. and Shenolikar, S. (1998) Front. Biosci. 3:961-972).

As used herein, the term “PP2AA CTLA4-interacting domain” includes a region of PP2AA that interacts with CTLA4. In a preferred embodiment, a PP2AA CTLA4-interacting domain interacts with the lysine rich motif of CTLA4. In another preferred embodiment, a PP2AA CTLA4-interacting domain comprises amino acid residues 392-589 of PP2AA (e.g., amino acid residues 392-589 of SEQ ID NO:7 or SEQ ID NO:9.

As used herein, the term “contacted with” includes exposure to, e.g., the exposure of cells or molecules to a test compound.

As used herein, the term “modulating” means changing or altering, and embraces both upmodulating and downmodulating.

As used herein, the term “immune cell” includes cells that are of hematopoietic origin and that play a role in the immune response. Immune cells include lymphocytes, such as B cells and T cells; natural killer cells; and myeloid cells, such as monocytes, macrophages, eosinophils, mast cells, basophils, and granulocytes.

As used herein, the term “T cell” includes CD4⁺ T cells and CD8⁺ T cells. The term T cell also includes both T helper 1 (Th1) type T cells and T helper 2 (Th2) type T cells. The term T cells preferably includes activated T cells and memory T cells. In a preferred embodiment, the T cells of the invention are memory T cells. The terms “antigen presenting cell” and “APC”, as used interchangeably herein, include professional antigen presenting cells (e.g., B lymphocytes, monocytes, dendritic cells, and Langerhans cells) as well as other antigen presenting cells (e.g., keratinocytes, endothelial cells, astrocytes, fibroblasts, and oligodendrocytes).

As used herein, the term “immune response” includes T cell-mediated and/or B cell-mediated immune responses that are influenced by modulation of T cell activation. Exemplary immune responses include B cell responses (e.g., antibody production), T cell responses (e.g., cytokine production and cellular cytotoxicity), and activation of cytokine responsive cells, e.g., macrophages. In a preferred embodiment of the invention, an immune response is T cell mediated. As used herein, the term “downmodulation” with reference to the immune response includes a diminution in any one or more immune responses, preferably T cell responses, while the term “upmodulation” with reference to the immune response includes an increase in any one or more immune responses, preferably T cell responses. It will be understood that upmodulation of one type of immune response may lead to a corresponding downmodulation in another type of immune response. For example, upmodulation of the production of certain cytokines (e.g., IL-10) can lead to downmodulation of cellular immune responses.

As used herein, the term “costimulatory receptor” includes receptors which transmit a costimulatory signal to a immune cell, e.g., CD28 or ICOS. As used herein, the term “inhibitory receptors” includes receptors which transmit a negative signal to an immune cell (e.g., CTLA4 or PD-1).

As used herein, the term “costimulate”, with reference to activated immune cells, includes the ability of a costimulatory molecule to provide a second, non-activating, receptor-mediated signal (a “costimulatory signal”) that induces proliferation or effector function. For example, a costimulatory signal can result in cytokine secretion, e.g., in a T cell that has received a T cell-receptor-mediated signal. Immune cells that have received a cell receptor-mediated signal, e.g., via an activating receptor, are referred to herein as “activated immune cells.”

As used herein, the term “activating receptor” includes immune cell receptors that bind antigen, complexed antigen (e.g., in the context of MHC molecules), or antibodies. Such activating receptors include T cell receptors (TCRs), B cell receptors (BCRs), cytokine receptors, LPS receptors, complement receptors, and Fc receptors.

For example, T cell receptors are present on T cells and are associated with CD3 molecules. T cell receptors are stimulated by antigen in the context of MHC molecules (as well as by polyclonal T cell activating reagents). T cell activation via the TCR results in numerous changes, e.g., protein phosphorylation, membrane lipid changes, ion fluxes, cyclic nucleotide alterations, RNA transcription changes, protein synthesis changes, and cell volume changes.

With respect to T cells, transmission of a costimulatory signal to a T cell involves a signaling pathway that is not inhibited by cyclosporin A. In addition, a costimulatory signal can induce cytokine secretion (e.g., IL-2 and/or IL-10) in a T cell and/or can prevent the induction of unresponsiveness to antigen, the induction of anergy, or the induction of cell death in the T cell.

As used herein, the term “inhibitory signal” refers to a signal transmitted via an inhibitory receptor (e.g., CTLA4 or PD-1) on a immune cell. Such a signal antagonizes a signal via an activating receptor (e.g., via a TCR, CD3, BCR, or Fc molecule) and can result, e.g., in inhibition of: second messenger generation; proliferation; or effector function in the immune cell, e.g., reduced phagocytosis, antibody production, or cellular cytotoxicity, or the failure of the immune cell to produce mediators (such as cytokines (e.g., IL-2) and/or mediators of allergic responses); or the development of anergy.

As used herein, the term “unresponsiveness” includes refractivity of immune cells to stimulation, e.g., stimulation via an activating receptor or a cytokine. Unresponsiveness can occur, e.g., because of exposure to immunosuppressants or high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD3 mediated signal) in the absence of a second signal (a costimulatory signal). Under these conditions, reexposure of the cells to the same antigen (even if reexposure occurs in the presence of a costimulatory molecule) results in failure to produce cytokines and, thus, failure to proliferate. Anergic T cells can, however, mount responses to unrelated antigens and can proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134).

With respect to CTLA4, the term “activity” includes the ability of a CTLA4 polypeptide to modulate an inhibitory signal in an activated immune cell, e.g., by engaging a natural ligand such as B7-1 or B7-2 on an antigen presenting cell. Modulation of an inhibitory signal in an immune cell results in modulation of proliferation of and/or cytokine secretion by an immune cell. CTLA4 can also modulate a costimulatory signal by competing with a costimulatory receptor for binding of its natural ligand(s), e.g., B7-1 or B7-2. Thus, the term “CTLA4 activity” includes the ability of a CTLA4 polypeptide to bind its natural ligand(s), e.g., B7-1 or B7-2, the ability to modulate immune cell costimulatory or inhibitory signals, and the ability to modulate the immune response.

In another embodiment, a “CTLA4 activity” includes the ability of CTLA4, e.g., the cytoplasmic domain of CTLA4, to interact with PP2AA in the cytoplasm of a cell. In a preferred embodiment, CTLA4 interacts with PP2AA via the lysine rich motif (SEQ ID NO:1) in the cytoplasmic domain of CTLA4.

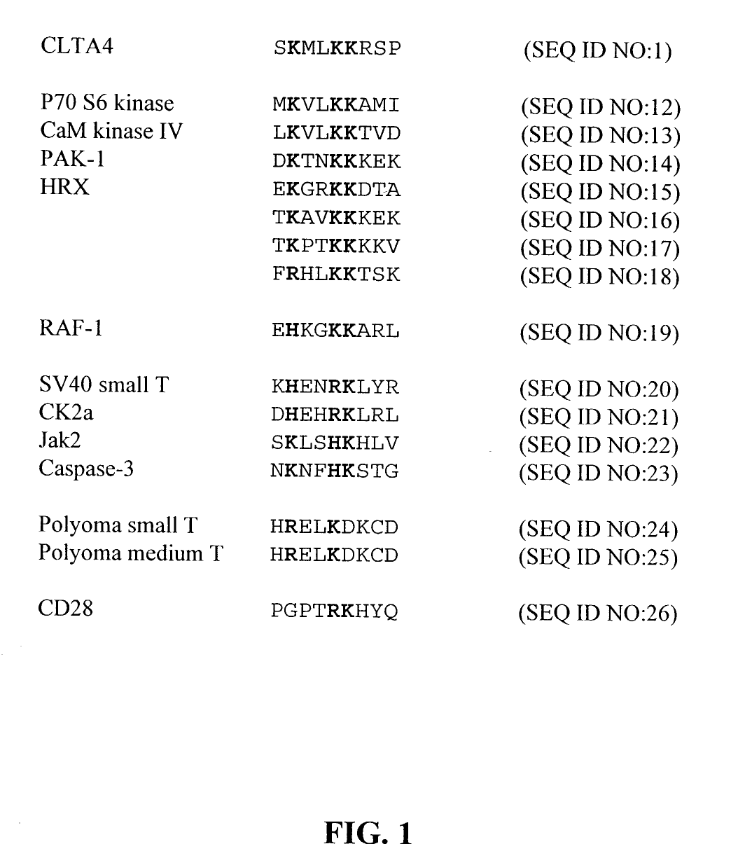

As used herein, a “lysine rich motif” is a conserved sequence motif found in proteins that bind to PP2AA (see FIG. 1 and Example 5). A lysine rich motif is involved in mediating the interaction between CTLA4 (e.g., the cytoplasmic domain of CTLA4) and PP2AA (e.g., amino acid residues 392-589 of PP2AA). The consensus sequence for a lysine rich motif, as determined herein, is X-[K/R/H]-X-X-[K/R/H]-K-X-X-X. As used herein, the letter “X” in the consensus sequence signifies any amino acid residue at the indicated position, the notation [K/R/H] signifies any one of K (lysine), R (arginine), or H (histidine) at the indicated position, and the one-letter codes for the amino acid residues are used according the to the IUPAC standard. In a preferred embodiment, a lysine rich motif is contained within a CTLA4 cytoplasmic domain. In another preferred embodiment, a CTLA4 lysine rich motif has the amino acid sequence SKMLKKRSP (SEQ ID NO:1). In still another preferred embodiment, a lysine rich motif is found at about amino acid residues 187-195 of SEQ ID NO:3 and at about amino acid residues 187-195 of SEQ ID NO:5.

Exemplary agents that modulate the interaction between a molecule comprising a CTLA4 lysine rich motif and a molecule comprising a PP2AA CTLA4-interacting domain is a peptidomimetic or a small molecule. Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986) Adv. Drug Res. 15:29; Veber and Freidinger (1985) TINS p.392; and Evans et al. (1987) J. Med. Chem. 30:1229, which are incorporated herein by reference) and are usually developed with the aid of computerized molecular modeling. Peptide mimetics that are structurally similar to CTLA4 lysine rich motifs or PP2AA CTLA4-interacting domains, or functional variants thereof, can be used to produce an equivalent product to the peptide agents described herein. Generally, peptidomimetics are structurally similar to the paradigm polypeptide but have one or more peptide linkages optionally replaced by a linkage selected from the group consisting of: —CH2NH—, —CH2S—, CH2—CH2—, —CH═CH— (cis and trans), —COCH2—, —CH(OH)CH2—, and —CH2SO—. This is accomplished by the skilled practitioner by methods known in the art which are further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J Pept. Prot. Res. 14:177-185 (—CH2NH—, CH2CH2—); Spatola, A. F. et al. (1986) Life Sci. 38:1243-1249 (—CH2—S); Hann, M. M. (1982) J. Chem. Soc. Perkin Trans. I. 307-314 (—CH—CH—, cis and trans); Almquist, R. G. et al. (190) J. Med Chem. 23:1392-1398 (—COCH2—); Jennings-White, C. et al. (1982) Tetrahedron Lett. 23:2533 (—COCH2—); Szelke, M. et al. European Appln. EP 45665 (1982) CA: 97:39405 (1982)(—CH(OH)CH2—); Holladay, M. W. et al. (1983) Tetrahedron Lett. (1983) 24:4401-4404 (—C(OH)CH2—); and Hruby, V. J. (1982) Life Sci. (1982) 31:189-199 (—CH2—S—); each of which is incorporated herein by reference. A particularly preferred non-peptide linkage is —CH2NH—. Such peptide mimetics may have significant advantages over polypeptides, including, for example: more economical production, greater chemical stability, enhanced pharmacological properties (half-life, absorption, potency, efficacy, etc.), altered specificity (e.g., a broad-spectrum of biological activities), and reduced antigenicity.

The term “small molecule” is a term of art and included molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, nucleic acids, carbohydrates, small organic molecules (e.g., polyketides) (Cane et al. 1998. Science 282:63), and natural product extract libraries. In another embodiment, the compounds are small, organic non-peptidic compounds. In a further embodiment, a small molecule is not biosynthetic.

The term “nucleic acid” as used herein is intended to include fragments or equivalents thereof (e.g., fragments or equivalents thereof of CTLA4 or PP2AA). The term “equivalent” is intended to include nucleic acid molecules comprising nucleotide sequences encoding functionally equivalent CTLA4 proteins, i.e., proteins which have the ability to bind to the natural ligand(s) of the CTLA4 antigen on immune cells, such as B7-1 and/or B7-2 on B cells, and inhibit (e.g., block) or interfere with immune cell mediated responses. In a preferred embodiment, a functionally equivalent CTLA4 protein has the ability to bind PP2AA in the cytoplasm of an immune cell, e.g., a T cell.

The term “isolated” as used herein refers to a nucleic acid or polypeptide molecules substantially free of cellular material or culture medium when produced by recombinant DNA techniques, or chemical precursors or other chemicals when chemically synthesized. An isolated nucleic acid molecule is also free of sequences which naturally flank the nucleic acid molecule (i.e., sequences located at the 5′ and 3′ ends of the nucleic acid molecule) in the organism from which the nucleic acid molecule is derived.

The nucleic acid molecules of the invention can be prepared by standard recombinant DNA techniques. A nucleic acid molecule of the invention can also be chemically synthesized using standard techniques. Various methods of chemically synthesizing polydeoxynucleotides are known, including solid-phase synthesis which has been automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein).

The CTLA4 and PP2A nucleic acid and polypeptides used in the invention are described in further detail below:

I. Nucleic Acid and Polypeptide Molecules Used in the Methods of the Invention

A. CTLA4

One embodiment of the invention features the use of an isolated nucleic acid molecule encoding a peptide having a CTLA4 activity and/or the use of a peptide having a CTLA4 activity. The phrase “peptide having a CTLA4 activity” or “peptide having an activity of CTLA4” is used herein to refer to a peptide having at least one biological activity of the CTLA4 protein, e.g., the ability to bind to the natural ligand(s) of the CTLA4 antigen on immune cells, such as B7-1 and/or B7-2 on B cells, or other known or as yet undefined ligands on immune cells, and transmit a negative signal to T cells. In a preferred embodiment, a CTLA4 activity is the ability to bind to PP2AA in the cytoplasm of a cell. In a more preferred embodiment, a CTLA4 activity is the ability to bind to PP2AA in the cytoplasm of a cell via the lysine rich motif of CTLA4 (SEQ ID NO:1).

In one embodiment, the CTLA4 protein is a human CTLA4 protein, the nucleotide and amino acid sequences of which are disclosed in Harper, K. et al. (1991) J. Immunol. 147:1037-1044 and Dariavach et al. (1988) Eur. J. Immunol. 18(12):1901-1905. The human CTLA4 nucleotide and amino acid sequences can also be accessed using GenBank Accession Nos. NM_{—}005214 and P16410, respectively. The human CTLA4 nucleotide and amino acid sequences are further set forth as SEQ ID NO:2 and SEQ ID NO:3, respectively. In another embodiment, the peptide having a CTLA4 activity binds B7-1 and/or B7-2 and comprises at least a portion of a cytoplasmic domain of the CTLA4 protein.

In another embodiment, the CTLA4 protein is a mouse CTLA4 protein, the nucleotide and amino acid sequences of which are disclosed in Brunet, J. F. et al. (1987) Nature 328:267-270. The mouse CTLA4 nucleotide and protein sequences can also be accessed using GenBank Accession Nos. NM_{—}009843 and P09793, respectively. The mouse CTLA4 nucleotide and amino acid sequences are further set forth as SEQ ID NO:4 and SEQ ID NO:5, respectively.

In another embodiment, the peptide having a CTLA4 activity binds B7-1, B7-2, and/or PP2AA, and comprises at least a portion of a cytoplasmic domain of the CTLA4 protein. Preferably, a CTLA4 cytoplasmic domain comprises amino acid residues 187-223 of the human CTLA4 protein (e.g., amino acid residues 187-223 of SEQ ID NO:3). In another embodiment, a CTLA4 cytoplasmic domain comprises amino acid residues 188-223 of the human CTLA4 protein (e.g., amino acid residues 188-223 of SEQ ID NO:3). In yet another embodiment, a CTLA4 cytoplasmic domain comprises amino acid residues 187-223 of the mouse CTLA4 protein (e.g., amino acid residues 187-223 of SEQ ID NO:5). In still another embodiment, a CTLA4 cytoplasmic domain comprises amino acid residues 188-223 of the mouse CTLA4 protein (e.g., amino acid residues 188-223 of SEQ ID NO:5). In a preferred embodiment, a CTLA4 cytoplasmic domain includes a lysine rich motif (SEQ ID NO:1), as described herein. In another embodiment, a CTLA4 includes one or more than one lysine rich motifs. CTLA4 proteins from other species (e.g., monkey or Drosophila) are also encompassed by the invention.

In another embodiment, a CTLA4 peptide binds PP2AA but does not bind B7-1 or B7-2 and is not anchored in the plasma membrane, e.g., does not comprise an extracellular domain or a transmembrane domain. Such a CTLA4 peptide may comprise the entire cytoplasmic domain of CTLA4 or a portion thereof. In one embodiment, a CTLA4 peptide may consist solely of a lysine rich motif (SEQ ID NO:1). In another embodiment, a CTLA4 peptide may comprise at least one lysine rich motif, e.g., may comprise one or more lysine rich motifs. CTLA4 peptides with multiple lysine rich motifs may facilitate identification of compounds that bind to a lysine rich motif by increasing the effective concentration of lysine rich motifs available for binding.

In another embodiment, a CTLA4 peptide may comprise a mutated lysine rich motif, e.g., as described in the Example section. For example, at least one or more of the lysines in the lysine rich motif (e.g., the lysines at the second, fifth, and/or sixth positions of SEQ ID NO:1) may be mutated to an alternate amino acid residue (e.g., alanine), such that binding of the lysine rich motif, and thus binding of the CTLA4 peptide in which it is contained, to PP2AA is reduced or eliminated. In a preferred embodiment, all three lysine residues are mutated to alanine. Methods for altering amino acid residues in such a manner are described herein and are well known in the art.

B. PP2AA

Another embodiment of the invention features the use of an isolated nucleic acid molecule encoding a peptide having a PP2AA activity and/or the use of a peptide having a PP2AA activity. The phrase “peptide having a PP2AA activity” or “peptide having an activity of PP2AA” is used herein to refer to a peptide having at least one biological activity of the PP2AA protein, e.g., the ability to remove phosphate groups from proteins, modulate the cell cycle, signal transduction, cytoskeleton organization, immediate early gene transcription, protein biosynthesis, and/or cholesterol biosynthesis, interact with a PP2AB and/or a PP2AC subunit, bind to the cytoplasmic domain of CTLA4, and/or bind to a lysine rich motif.

In one embodiment, the PP2AA protein is a human PP2AA protein, the amino acid sequence of which can be found using GenBank Accession Nos. P30153, A34541, AAA36399, and/or NP_{—}055040, and which is further set forth as SEQ ID NO:7. The nucleotide sequence of human PP2AA can be found using GenBank Accession Nos. J02902, M65254, NM_{—}014225, and/or NM_{—}002716, and is further set forth as SEQ ID NO:6. In another embodiment, the peptide having a PP2AA activity binds CTLA4 and comprises at least amino acid residues 392-589 of PP2AA (e.g., amino acid residues 392-589 of SEQ ID NO:7).

In another embodiment, the PP2AA protein is a mouse PP2AA protein, the amino acid sequences of which can be found using GenBank Accession Nos. BAA75478 and NP_{—}058587, and which is set forth as SEQ ID NO:9. The mouse PP2AA nucleotide sequence can be found using GenBank Nos. NM_{—}016891 and/or AB021743, and is further set forth as SEQ ID NO:8. In another embodiment, the peptide having a PP2AA activity binds CTLA4 and comprises at least amino acid residues 392-589 of PP2AA (e.g., amino acid residues 392-589 of SEQ ID NO:9).

PP2AA proteins from other species (e.g., monkey or Drosophila) are also encompassed by the invention.

The nucleic acids used in the methods of the invention can be DNA or RNA. Nucleic acid encoding a peptide having a CTLA4 activity or a PP2AA activity may be obtained from mRNA present in activated T lymphocytes. It is also possible to obtain nucleic acid encoding CTLA4 or PP2AA from genomic DNA, e.g., T cell genomic DNA. For example, the genes encoding CTLA4 or PP2AA can be cloned from either a cDNA or a genomic library in accordance with standard protocols. A cDNA encoding CTLA4 or PP2AA can be obtained by isolating total mRNA from an appropriate cell line. Double stranded cDNAs can then prepared from the total mRNA. Subsequently, the cDNAs can be inserted into a suitable plasmid or bacteriophage vector using any one of a number of known techniques. Genes encoding CTLA4 and PP2AA can also be cloned using established polymerase chain reaction techniques in accordance with the nucleotide sequence information known in the art (and/or as described herein). For example, a DNA vector containing a CTLA4 or PP2AA cDNA can be used as a template in PCR reactions using oligonucleotide primers designed to amplify a desired region of the CTLA4 or PP2AA cDNA, e.g., the CTLA4 cytoplasmic domain, to obtain an isolated DNA fragment encompassing this region using standard techniques.

It will be appreciated by those skilled in the art that various modifications and equivalents of the nucleic acids encoding the CTLA4 and PP2AA peptides exist. For example, different cell lines can be expected to yield DNA molecules having different sequences of bases. Additionally, variations may exist due to genetic polymorphisms or cell-mediated modifications of the genetic material. Furthermore, the nucleotide sequence encoding a CTLA4 or PP2AA peptide can be modified by genetic techniques to produce proteins with altered amino acid sequences that retain the functional properties of CTLA4 (e.g., the ability to bind to B7-1, B7-2, and/or PP2AA) or PP2AA (e.g., the ability to remove a phosphate group from a protein or the ability to bind to CTLA4 or a lysine rich motif). Such sequences are considered within the scope of the invention, wherein the expressed protein is capable of binding e.g., CTLA4 (in the case of PP2AA) or PP2AA (in the case of CTLA4) and, when in the appropriate form can inhibit T cell activation and modulate immune responses and immune function.

To express a CTLA4 or PP2AA peptide, the nucleotide sequence encoding the CTLA4 or PP2AA peptide may includes a nucleotide sequence encoding a signal sequence which, upon transcription and translation of the nucleic acid molecule, directs secretion or membrane targeting of the peptide. A native CTLA4 signal sequence (e.g., the human CTLA4 signal sequence disclosed in Harper, K. et al. (1991) J. Immunol. 147:1037-1044) can be used or alternatively, a heterologous signal sequence can be used. For example, the oncostatin-M signal sequence (Malik N. et al.(1989) Mol. Cell. Biol. 9(7):2847-2853) or an immunoglobulin signal sequence can be used to direct secretion or membrane targeting of a CTLA4 or PP2AA peptide. A nucleotide sequence encoding a signal sequence can be incorporated into the CTLA4 or PP2A gene by standard recombinant DNA techniques, such as by “zip up” PCR or by ligating a nucleic acid fragment encoding the signal sequence in-frame at the 5′ end of a nucleic acid fragment encoding CTLA4 or PP2AA.

It will also be appreciated by those skilled in the art that the peptides used in the methods of the invention may be chemically synthesized by standard methods known in the art.

II. Expression Vectors and Host Cells

The CTLA4 and PP2AA peptides used in the methods of the invention can be expressed by incorporating a CTLA4 or PP2AA gene described herein into an expression vector and introducing the expression vector into an appropriate host cell. Accordingly, the invention further pertains to the use of expression vectors containing a nucleic acid encoding a CTLA4 or PP2AA peptide and to host cells into which such expression vectors have been introduced. An expression vector of the invention, as described herein, typically includes nucleotide sequences encoding the CTLA4 peptide operably linked to at least one regulatory sequence. As used interchangeably herein, the terms “operably linked” and “operatively linked” are intended to mean that the nucleotide sequence is linked to a regulatory sequence in a manner which allows expression of the nucleotide sequence in a host cell (or by a cell extract). Regulatory sequences are art-recognized and can be selected to direct expression of the desired protein in an appropriate host cell. The term regulatory sequence is intended to include promoters, enhancers, polyadenylation signals and other expression control elements. Such regulatory sequences are known to those skilled in the art and are described in Goeddel, Gene Expression Technology: Methods in Enzymology 185, Academic Press, San Diego, Calif. (1990). It should be understood that the design of the expression vector may depend on such factors as the choice of the host cell to be transfected and/or the type and/or amount of protein desired to be expressed.

In a preferred embodiment, the CTLA4 and/or PP2AA nucleic acid molecules are operably linked to regulatory sequences which allow their expression to be controlled by the addition or removal of an exogenous compound, e.g., tetracycline, as described herein in the Example section. Methods and sequences relating to the use of tetracycline controlled regulatory sequences can be found, for example, in Gossen, M. and Bujard, H (1992) Proc. Natl. Acad. Sci. USA 89(12):5547-51; Gossen, M. et al. (1993) Trends Biochem. Sci. 18(12):471-5; Gossen, M. et al. (1994) Curr. Opin. Biotechnol. 18(12):471-5; Shockett, P. et al. (1995) Proc. Natl. Acad. Sci. USA 92(14):6522-6; Baron U. et al. (1995) Nucleic Acids Res. 23(17):3605-6; Lang, Z. and Feingold, J. M. (1996) Gene 168(2):169-71; Hofmann, A. et al. (1996) Proc. Natl. Acad. Sci. USA 93(11):5185-90; O'Brien, K. et al. (1997) Gene 184(1):115-20; Lindemann, D. et al. (1997) Mol. Med. 3(7):466-76; Baron, U. et al. (1997) Nucleic Acids Res. 25(14):2723-9; Bujard, H. (1999) J. Gene Med. 1(5):372-4; and Freundlieb, S. et al. (1999) J. Gene Med. 1(1):4-12.

An expression vector of the invention can be used to transfect cells, either prokaryotic or eukaryotic (e.g., mammalian, insect or yeast cells) to thereby produce peptides encoded by nucleotide sequences of the vector. Expression in prokaryotes is most often carried out in E. coli with vectors containing constitutive or inducible promoters. Certain E. coli expression vectors (so called fusion-vectors) are designed to add a number of amino acid residues to the expressed recombinant protein, usually to the amino terminus of the expressed protein. Such fusion vectors typically serve three purposes: 1) to increase expression of recombinant protein; 2) to increase the solubility of the target recombinant protein; and 3) to aid in the purification of the target recombinant protein by acting as a ligand in affinity purification. Examples of fusion expression vectors include pGEX (Amrad Corp., Melbourne, Australia; also available from Pharmacia Corp.) and pMAL (New England Biolabs, Beverly, Mass.) which fuse glutathione S-transferase and maltose E binding protein, respectively, to the target recombinant protein. Accordingly, a CTLA4 or PP2AA gene may be linked to additional coding sequences in a prokaryotic fusion vector to aid in the expression, solubility or purification of the fusion protein. Often, in fusion expression vectors, a proteolytic cleavage site is introduced at the junction of the fusion moiety and the target recombinant protein to enable separation of the target recombinant protein from the fusion moiety subsequent to purification of the fusion protein. Such enzymes, and their cognate recognition sequences, include Factor Xa, thrombin and enterokinase.

Inducible non-fusion expression vectors include pTrc (Amann et al., (1988) Gene 69:301-315) and pET 1d (Studier et al., Gene Expression Technology: Methods in Enzymology 185, Academic Press, San Diego, Calif. (1990) 60-89). Target gene expression from the pTrc vector relies on host RNA polymerase transcription from the hybrid trp-lac fusion promoter. Target gene expression from the pET 11d vector relies on transcription from the T7 gn 10-lac 0 fusion promoter mediated by a coexpressed viral RNA polymerase (T7 gn1). This viral polymerase is supplied by host strains BL21(DE3) or HMS 174(DE3) from a resident λ prophage harboring a T7 gnl under the transcriptional control of the lacUV 5 promoter.

One strategy to maximize expression of recombinant CTLA4 or PP2AA peptide in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy would be to alter the nucleotide sequence of the CTLA4 or PP2AA peptide to be inserted into an expression vector so that the individual codons for each amino acid would be those preferentially utilized in highly expressed E. coli proteins (Wada et al., (1992) Nuc. Acids Res. 20:2111-2118). Such alteration of nucleic acid sequences are encompassed by the invention and can be carried out by standard DNA synthesis techniques.

In another embodiment, a CTLA4 or PP2AA peptide can be expressed in a eukaryotic host cell, such as mammalian cells (e.g., T cells such as Jurkat cells, COS cells, Chinese hamster ovary cells (CHO) or NSO cells), insect cells (e.g., using a baculovirus vector) or yeast cells. In a preferred embodiment, a eukaryotic host cell is a Jurkat T cell. Other suitable host cells may be found in Goeddel, (1990) supra or are known to those skilled in the art. Eukaryotic, rather than prokaryotic, expression of a CTLA4 or PP2AA peptide may be preferable since expression of eukaryotic proteins in eukaryotic cells can lead to partial or complete glycosylation and/or formation of relevant inter- or intra-chain disulfide bonds of a recombinant protein. For expression in mammalian cells, the expression vector's control functions are often provided by viral material. For example, commonly used promoters are derived from polyoma, Adenovirus 2, cytomegalovirus and Simian Virus 40. To express a CTLA4 peptide in mammalian cells, generally COS cells (Gluzman, Y., (1981) Cell 23:1751-182) are used in conjunction with such vectors as pCDM8 (Seed, B., (1987) Nature 329:840) for transient amplification/expression, while CHO (dhfr⁻ Chinese Hamster Ovary) cells are used with vectors such as pMT2PC (Kaufman et al. (1987), EMBO J 6:187-195) for stable amplification/expression in mammalian cells. A preferred cell line for production of recombinant protein is the NSO myeloma cell line available from the ECACC (catalog #85110503) and described in Galfre, G. and Milstein, C. ((1981) Methods in Enzymology 73(13):3-46; and Preparation of Monoclonal Antibodies: Strategies and Procedures, Academic Press, N.Y., N.Y). Examples of vectors suitable for expression of recombinant proteins in yeast (e.g., S. cerivisae) include pYepSec1 (Baldari. et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Baculovirus vectors available for expression of proteins in cultured insect cells (SF 9 cells) include the pAc series (Smith et al., (1983) Mol. Cell Biol. 3:2156-2165) and the pVL series (Lucklow, V. A., and Summers, M. D., (1989) Virology 170:31-39).

Vector DNA can be introduced into prokaryotic or eukaryotic cells via conventional transformation or transfection techniques such as calcium phosphate or calcium chloride coprecipitation, DEAE-dextran-mediated transfection, lipofection, or electroporation. Suitable methods for transforming host cells can be found in Sambrook et al. (Molecular Cloning: A Laboratory Manual, 2nd Edition, Cold Spring Harbor Laboratory press (1989)), and other laboratory textbooks.

For stable transfection of mammalian cells, it is known that, depending upon the expression vector and transfection technique used, only a small faction of cells may integrate DNA into their genomes. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker may be introduced into a host cell on the same plasmid as the gene of interest or may be introduced on a separate plasmid. Cells containing the gene of interest can be identified by drug selection (e.g., cells that have incorporated the selectable marker gene will survive, while the other cells die). The surviving cells can then be screened for production of CTLA4 or PP2AA peptides by, for example, Western blotting or immunoprecipitation from cell supernatant with an anti-CTLA4 or anti-PP2AA monoclonal antibody.

The invention also features methods of producing CTLA4 and PP2AA peptides. For example, a host cell transfected with a nucleic acid vector directing expression of a nucleotide sequence encoding a CTLA4 or PP2AA peptide can be cultured in a medium under appropriate conditions to allow expression of the protein to occur. In one embodiment, a recombinant expression vector containing DNA encoding a CTLA4 peptide is produced. In another embodiment, a recombinant expression vector containing DNA encoding a PP2A peptide is produced. Peptides produced by recombinant technique may be secreted and isolated from a mixture of cells and medium containing the protein. Alternatively, the protein may be retained cytoplasmically and the cells harvested, lysed and the protein isolated. A cell culture typically includes host cells, media and other byproducts. Suitable mediums for cell culture are well known in the art. Protein can be isolated from cell culture medium, host cells, or both using techniques known in the art for purifying proteins. Alternative to recombinant expression, a CTLA4 or PP2A peptide can be synthesized chemically using standard peptide synthesis techniques known in the art.

III. Screening Assays

Modulators of the interaction between CTLA4 and PP2AA can be known (e.g., dominant negative inhibitors of CTLA4/PP2AA interaction, intracellular antibodies that interfere with the interaction between CTLA4/PP2AA, peptide inhibitors derived from CTLA4 or PP2AA) or can be identified using the methods described herein. The invention provides a method (also referred to herein as a “screening assay”) for identifying other modulators, i.e., candidate or test compounds or agents (e.g., a plurality of compounds such as, peptidomimetics, small molecules or other drugs) which modulate the interaction between CTLA4 and PP2AA and for testing or optimizing the activity of other agents.

In a preferred embodiment, the invention provides assays for screening candidate or test compounds which bind to the lysine-rich motif of CTLA4, and thus modulate the ability of the CTLA4 polypeptide to interact with PP2AA via the lysine-rich motif. In another preferred embodiment, the invention provides assays for screening candidate or test compounds which have a stimulatory or inhibitory effect on the interaction between CTLA4 and PP2AA.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad Sci. U.S. 90:6909; Erb et al. (1994) Proc. Natl. Acad. Sci. USA 91:11422; Zuckermann et al. (1994) J. Med Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad Sci. USA 87:6378-6382; Felici (1991)J. Mol. Biol. 222:301-310; Ladner supra.).

In many drug screening programs which test libraries of modulating agents and natural extracts, high throughput assays are desirable in order to maximize the number of modulating agents surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test modulating agent. Moreover, the effects of cellular toxicity and/or bioavailability of the test modulating agent can be generally ignored in the in vitro system, the assay instead being focused primarily on the effect of the drug on the molecular target as may be manifest in an alteration of binding affinity with upstream or downstream elements. Methods and services for high-throughput drug screening can be found commercially, for example, from companies such as GPC Biotech (Martinsried, Germany; Cambridge, Mass.; and Princeton, N.J.), Upstate Discovery (Dundee, Scotland), Beckman Coulter, Inc. (Fullerton, Calif.), Transtech Pharma (High Point, N.C.), Morphochem (Monmouth Junction, N.J.), Pall Corporation (Fishers, Ind.), Torcon Instruments, Inc. (Torrance, Calif.), Axys Pharmaceuticals, Inc. (South San Francisco, Calif.), Biotium, Inc. (Hayward, Calif.), The High Throughput Screening Teaching, Research, and Training Facility at Rutgers University (NJ), Zymark Corporation, Invitrotech (Baltimore, Md.), Applied Biosystems (Foster City, Calif.), Pharmacopeia, Inc. (Princeton, N.J.), 3Dimensional Pharmaceuticals, Inc. (Exton, Pa.), Prozyme (San Leandro, Calif.), The Automation Partnership (Hertfordshire, UK), BioLeads (Heidelberg, Germany), Polymer Laboratories (Amherst, Mass. and other locations), Luminex Corporation, and many other companies.

The identification of PP2AA as a binding partner of CTLA4 described for the first time herein makes assays that can be used to screen for modulating agents which are either agonists or antagonists of the normal cellular function of the subject CTLA4 polypeptides, e.g., those that modulate the interaction between CTLA4 and PP2AA, e.g., via the lysine rich motif possible. For example, the invention provides a method in which an indicator composition is provided which has a CTLA4 peptide having a CTLA4 activity. The CTLA4 peptide can be a full-length CTLA4 polypeptide, a CTLA4 cytoplasmic domain, a fragment of a CTLA4 cytoplasmic domain, or a peptide consisting solely of a lysine rich motif. The CTLA4 peptide can also be a peptide containing more than one lysine rich motif. Such a peptide may be useful, for example, in identifying compounds that bind to the lysine rich motif because they may increase the effective concentration of lysine rich motifs available for the compound to bind to. The indicatory composition may also comprise a PP2AA peptide, e.g., a full-length PP2AA peptide or a fragment thereof, for example amino acid residues 392-589 of PP2AA. The indicator composition can be contacted with a test compound. The effect of the test compound on CTLA4 activity, as measured by a change in the indicator composition, can then be determined to thereby identify a compound that modulates the ability of a CTLA4 protein to interact with a PP2AA protein. A statistically significant change, such as a decrease or increase, in the level of CTLA4 activity or in the level of interaction between CTLA4 and PP2AA in the presence of the test compound (relative to what is detected in the absence of the test compound) is indicative of the test compound being a modulating agent of CTLA4-PP2AA interaction. In one preferred embodiment, the agent binds to the lysine rich motif of CTLA4. The indicator composition can be, for example, a cell or a cell extract. In one embodiment, CTLA4-PP2AA interaction is assessed as described in the appended Examples. In a preferred embodiment, CTLA4-PP2AA interaction is determined by the ability of the CTLA4 peptide to bind to PP2AA via the lysine rich motif. In another embodiment, CTLA4 activity is determined by the ability of CTLA4 to inhibit T cell activation.

In an exemplary screening assay of the present invention, the modulating agent of interest is contacted with PP2AA. To the mixture of the modulating agent and the interactor molecule is then added a composition containing a CTLA4 peptide. Detection and quantification of the interaction of CTLA4 (e.g., the lysine-rich motif of CTLA4) with PP2AA provide a means for determining a modulating agent's efficacy at inhibiting (or potentiating) complex formation between CTLA4 and PP2AA.

The efficacy of the modulating agent can be assessed by generating dose response curves from data obtained using various concentrations of the test modulating agent. Moreover, a control assay can also be performed to provide a baseline for comparison. In an exemplary control assay, isolated and purified CTLA4 peptide is added to a composition containing PP2AA, and the formation of a complex is quantitated in the absence of the test modulating agent.

In one embodiment, an assay is a cell-based assay in which a cell (e.g., a T cell) which expresses a CTLA4 polypeptide or biologically active portion thereof is contacted with a test compound, and the ability of the test compound to modulate CTLA4-PP2AA interaction is determined. In a preferred embodiment, determining the ability of the test compound to modulate CTLA4-PP2AA interaction can be accomplished by monitoring, for example, the ability of CTLA4 to bind to PP2AA. Either or both of the CTLA4 and PP2AA polypeptides or portions thereof can be either exogenous to the cell (i.e., the cell can be caused to express the molecules, e.g., by transfection or transformation) or can be endogenous to the cell.

Determining the ability of the test compound to modulate CTLA4 binding to PP2AA can be accomplished, for example, by coupling PP2AA with a radioisotope or enzymatic label such that binding of the PP2AA to CTLA4 can be determined by detecting the labeled PP2AA in a complex. Alternatively, CTLA4 could be coupled with a radioisotope or enzymatic label to monitor the ability of a test compound to modulate CTLA4 binding to PP2AA in a complex. Determining the ability of the test compound to bind CTLA4 (e.g., to the lysine rich motif) can be accomplished, for example, by coupling the compound with a radioisotope or enzymatic label such that binding of the compound to CTLA4 can be determined by detecting the labeled CTLA4 compound in a complex. For example, compounds can be labeled with 125I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product. In a preferred embodiment, the ability of a test compound to modulate CTLA4 binding to PP2AA can be determined by measuring, in the absence or the presence of the compound, the amount of PP2AA bound to CTLA4 by immunoprecipitation, e.g., as described in the Examples section.

It is also within the scope of this invention to determine the ability of a compound to interact with CTLA4 (e.g., to interact with the lysine-rich motif) without the labeling of any of the interactants. For example, a microphysiometer can be used to detect the interaction of a compound with CTLA4 without the labeling of either the compound or the CTLA4 (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and CTLA4.

In another embodiment, an assay is a cell-based assay comprising contacting a cell expressing PP2AA with a test compound and determining the ability of the test compound to modulate (e.g., stimulate or inhibit) the activity of PP2AA. Determining the ability of the test compound to modulate the activity of PP2AA can be accomplished, for example, by determining the ability of a CTLA4 peptide to bind to or interact with the PP2AA.

Determining the ability of the CTLA4 peptide, or a biologically active fragment thereof (e.g., a peptide comprising the lysine-rich motif of SEQ ID NO:1), to bind to or interact with PP2AA, can be accomplished by one of the methods described above for determining direct binding. In a preferred embodiment, determining the ability of the CTLA4 peptide to bind to or interact with PP2AA can be accomplished by determining the activity of CTLA4. As described herein, the binding of PP2AA to CTLA4 downregulates CTLA4 activity (and thus upregulates T cell activity and/or immune responses). For example, the activity of CTLA4 can be determined by detecting T cell activation (using methods known in the art or described herein). In one embodiment, T cell activation can be determined by measuring T cell proliferation, using standard methods. In another embodiment, T cell activation can be determined by measuring cytokine production (e.g., IL-2 production) using a standard cytokine ELISA, a Western blot, or other methods known in the art. In another embodiment, T cell activation can be determined by detecting the induction of a reporter gene (comprising a target-responsive regulatory element such as the IL-2 promoter/enhancer region operatively linked to a nucleic acid encoding a detectable marker, e.g., luciferase).

In yet another embodiment, an assay of the present invention is a cell-free assay in which a CTLA4 peptide or biologically active portion thereof (e.g., a peptide comprising the lysine-rich motif of SEQ ID NO:1) is contacted with a test compound and the ability of the test compound to bind to the CTLA4 polypeptide or biologically active portion thereof is determined. Preferred biologically active portions of the CTLA4 polypeptides to be used in assays of the present invention include fragments which participate in interactions with PP2AA, e.g., at least a portion of a cytoplasmic domain which binds to PP2AA. In a preferred embodiment, a biologically active portion of CTLA4 comprises the lysine-rich motif of SEQ ID NO:1. In a particularly preferred embodiment, a biologically active portion of CTLA4 comprises at least one or more lysine-rich motifs. Binding of the test compound to the CTLA4 peptide can be determined either directly or indirectly as described above. In a preferred embodiment, the assay includes contacting the CTLA4 peptide or biologically active portion thereof with PP2AA to form an assay mixture, contacting the assay mixture with a test compound, and determining the ability of the test compound to interact with a CTLA4 peptide, wherein determining the ability of the test compound to interact with a CTLA4 peptide comprises determining the ability of the test compound to preferentially bind to CTLA4 or biologically active portion thereof (e.g., to the lysine rich motif) as compared to PP2AA.

In another embodiment, the assay is a cell-free assay in which a CTLA4 peptide or biologically active portion thereof (e.g., a peptide comprising the lysine-rich motif of SEQ ID NO:1) is contacted with a test compound and the ability of the test compound to modulate (e.g., stimulate or inhibit) the activity of the CTLA4 peptide or biologically active portion thereof is determined. Determining the ability of the test compound to modulate the activity of a CTLA4 polypeptide can be accomplished, for example, by determining the ability of the CTLA4 polypeptide to bind to PP2AA by one of the methods described above for determining direct binding. Determining the ability of the CTLA4 polypeptide to bind to PP2AA can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a CTLA4 polypeptide can be accomplished by determining the ability of the CTLA4 peptide to modulate the activity of PP2AA. For example, the activity of the PP2AA on an appropriate target (e.g., the ability of PP2AA to dephosphorylate an appropriate target) can be determined, or the binding of the PP2AA to an appropriate target can be determined as previously described.

The cell-free assays of the present invention are amenable to use of both soluble and/or membrane-bound forms of polypeptides (e.g., CTLA4 peptides or biologically active portions thereof). In the case of cell-free assays in which a membrane-bound form a polypeptide is used (e.g., a cell-surface CTLA4), it may be desirable to utilize a solubilizing agent such that the membrane-bound form of the polypeptide is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)n, 3-[(3-cholamidopropyl)dimethylamminio]-1-propane sulfonate (CHAPS), 3-[(3cholamidopropyl)dimethylamminio]-2-hydroxy-1-propane sulfonate (CHAPSO), or N-dodecyl═N,N-dimethyl-3-ammonio-1-propane sulfonate.

In more than one embodiment of the above assay methods of the present invention, it may be desirable to immobilize either CTLA4 or PP2AA to facilitate separation of complexed from uncomplexed forms of one or both of the polypeptides, as well as to accommodate automation of the assay. Binding of a test compound to a CTLA4 peptide, or interaction of a CTLA4 peptide with PP2AA in the presence and absence of a candidate compound can be accomplished in any vessel suitable for containing the reactants. Examples of such vessels include microtitre plates, test tubes, and micro-centrifuge tubes. In one embodiment, a fusion protein can be provided which adds a domain that allows one or both of the polypeptides to be bound to a matrix. For example, glutathione-S-transferase/CTLA4 fusion proteins or glutathione-S-transferase/PP2AA fusion proteins can be adsorbed onto glutathione sepharose beads (Sigma Chemical, St. Louis, Mo.) or glutathione derivatized microtitre plates, which are then combined with the test compound or the test compound and either the non-adsorbed PP2AA peptide or CTLA4 peptide, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of CTLA4-PP2AA binding or activity determined using standard techniques.

Other techniques for immobilizing polypeptides on matrices can also be used in the screening assays of the invention. For example, either a CTLA4 peptide or a PP2AA peptide can be immobilized utilizing conjugation of biotin and streptavidin. Biotinylated CTLA4 polypeptide or PP2AA can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with CTLA4 polypeptide or PP2AA but which do not interfere with binding of the CTLA4 peptide to PP2AA can be derivatized to the wells of the plate, and PP2AA or CTLA4 peptide is trapped in the wells by antibody conjugation. Methods for detecting such complexes, in addition to those described above for the GST-immobilized complexes, include immunodetection of complexes using antibodies reactive with the CTLA4 or PP2AA peptide, as well as enzyme-linked assays which rely on detecting an enzymatic activity associated with the CTLA4 or PP2AA peptide, either intrinsic or extrinsic activity. In the instance of the latter, the enzyme can be chemically conjugated or provided as a fusion protein with a CTLA4 binding protein (e.g., an anti-CTLA4 antibody. For example, the CTLA4 binding protein can be chemically cross-linked or genetically fused with horseradish peroxidase, and the amount of protein trapped in the complex can be assessed with a chromogenic substrate of the enzyme, e.g. 3,3′-diamino-benzadine terahydrochloride or 4-chloro-1-napthol. Likewise, a fusion protein comprising the protein and glutathione-S-transferase can be provided, and complex formation quantitated by detecting the GST activity using 1-chloro-2,4-dinitrobenzene (Habig et al., 1974, J. Biol. Chem 249:7130).

For processes which rely on immunodetection for quantitating one of the proteins trapped in the complex, antibodies against the protein, such as anti-CTLA4 antibodies or anti-PP2AA antibodies, can be used. Alternatively, the protein to be detected in the complex can be “epitope tagged” in the form of a fusion protein which includes, in addition to the CTLA4 sequence, a second protein for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al., 1991, J. Biol. Chem. 266:21150-21157) which includes a 10-residue sequence from c-myc, as well as the pFLAG system (International Biotechnologies, Inc.) or the pEZZ-protein A system (Pharamacia, N.J.).

In an alternative embodiment, determining the ability of the test compound to modulate the activity of and/or bind a CTLA4 peptide or modulate the interaction between CTLA4 and PP2AA can be accomplished by determining the ability of the test compound to modulate the activity of a molecule that functions downstream of or concomitantly with CTLA4, e.g., a T cell receptor (TCR). For example, levels of second messengers, the activity of the interacting molecule on an appropriate target, or the binding of the interactor to an appropriate target can be determined as previously described. In one embodiment, TCR associated tyrosine kinase activity can be determined. Other methods for determining the activity of a T cell receptor are known in the art.

In yet another aspect of the invention, the CTLA4 or PP2AA peptides can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify compounds (e.g., small molecules or other polypeptides) which can modulate the interaction of CTLA4 and PP2AA.

The two-hybrid system is based on the modular nature of most transcription factors, which consist of separable DNA-binding and activation domains. Briefly, the assay utilizes two different DNA constructs. In one construct, the gene that codes for a CTLA4 peptide is fused to a gene encoding the DNA binding domain of a known transcription factor (e.g., GAL-4). In the other construct, a PP2AA DNA sequence (e.g., a full length PP2AA sequence or a PP2AA peptide comprising amino acid residues 392-589 of PP2AA), referred to herein as the “prey” or “sample” is fused to a gene that codes for the activation domain of the known transcription factor. If the “bait” and the “prey” polypeptides are able to interact, in vivo, forming a CTLA4-PP2AA complex, the DNA-binding and activation domains of the transcription factor are brought into close proximity. This proximity allows transcription of a reporter gene (e.g., LacZ or a gene which confers survival on nutritionally selective media) which is operably linked to a transcriptional regulatory site responsive to the transcription factor.

In a preferred embodiment, a screening assay of the present invention utilizes the yeast cells such as those described in Example 1, wherein the cytoplasmic domain of CTLA4 is used as the “bait” and PP2AA (e.g., residues 392-589 of PP2AA) are used as the “prey”. The “bait” may also comprise any fragment of the CTLA4 cytoplasmic domain which includes a lysine-rich domain. In one embodiment, the bait may comprise at least one or more lysine rich domains. The prey may also comprise the full-length PP2AA amino acid sequence. In an alternate embodiment, the bait may comprise a PP2AA peptide, while the prey may comprise a CTLA4 peptide. In a preferred embodiment, yeast cells containing the CTLA4 bait and the PP2AA prey are cultured under conditions that allow for interaction of the bait and the prey (e.g., as described in the Example section). The cells are then contacted with a compound and the ability of the compound to modulate the interaction of the bait and the prey is determined. In one embodiment, interaction of the bait and prey is determined by the level growth on nutritionally selective media. In another embodiment, interaction of the bait and prey is determined by expression of a LacZ reporter gene. It will be understood by those skilled in the art that when using nutritional selection as a readout of the assay, compounds that inhibit the interaction of CTLA4 and PP2AA will prevent growth of the cells. Accordingly, a compound identified as being a modulator of CTLA2-PP2AA interaction under such conditions should also be tested for the ability to inhibit the growth of the cells under non-selective conditions, so that compounds that generally inhibit yeast growth will not be chosen for further study.

The present invention also provides a kit comprising a two-hybrid system having (1) a first hybrid protein comprising a CTLA4 peptide (e.g., a CTLA4 cytoplasmic domain or fragment thereof comprising at least one lysine-rich motif) and a transcriptional activation domain, and (2) a second hybrid protein comprising PP2AA and a DNA-binding domain, a host cell, and an instruction manual. Alternatively, the CTLA4 peptide may be fused to the DNA-binding domain and PP2AA fused to the activation domain. Such kits may optionally include a panel of agents for testing for the capacity to alter intermolecular binding between the first and second hybrid proteins.

In another aspect, the invention pertains to a combination of two or more of the assays described herein. For example, a modulating agent can be identified using a cell-based or a cell-free assay, and the ability of the agent to modulate CTLA4-PP2AA interaction can be confirmed in vivo, e.g., in an animal such as an animal model for an autoimmune or inflammatory disease or an organ transplant.

This invention further pertains to novel agents identified by the above-described screening assays. Accordingly, it is within the scope of this invention to further use an agent identified as described herein in an appropriate animal model. For example, an agent identified as described herein (e.g., an agent that can bind to a lysine-rich motif or an agent that can modulate the interaction of CTLA4 and PP2AA) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

CTLA4 and PP2AA peptides, especially those portions which form direct contacts in CTLA4/PP2AA heterodimers (e.g., a lysine-rich motif), can also be used for rational drug design of candidate CTLA4 modulating agents (e.g., molecules useful for downregulating CTLA4 activity, and thus, downregulating immune responses). The production of substantially pure CTLA4 peptide/PP2AA complexes and computational models which can be used for protein X-ray crystallography or other structure analysis methods, such as the DOCK program (Kuntz et al. (1982) J. Mol. Biol. 161:269; Kuntz, I.D. (1992) Science 257:1078) and variants thereof. Potential therapeutic drugs may be designed rationally on the basis of structural information thus provided. In one embodiment, such drugs are designed to prevent or enhance formation of a CTLA4 polypeptide:PP2AA complex. In another embodiment, such drugs are designed to bind to a lysine rich motif of CTLA4. Thus, the present invention may be used to design drugs, including drugs with a capacity to inhibit or promote binding of CTLA4 to PP2AA.

IV. Methods of Treatment:

The present invention provides for both prophylactic and therapeutic methods of treating a subject at risk of (or susceptible to) a disorder or having a disorder associated with an aberrant or unwanted immune response, e.g., an immune system disorder such as an autoimmune disorder, graft-versus-host disease (GVHD), or a tendency to have immune-mediated spontaneous abortions.

Modulatory methods of the invention involve contacting a cell (e.g., a T cell) with a agent that modulates the interaction of CTLA4 with PP2AA (e.g., via the lysine rich motif of CTLA4) e.g., an agent that binds to the lysine-rich motif. An agent that modulates CTLA4 binding to PP2AA can be an agent as described herein, such as a CTLA4 peptide (e.g., a peptide comprising a cytoplasmic domain of CTLA4 or a fragment thereof, or a peptide comprising or consisting of at least one lysine-rich motif of SEQ ID NO:1), a nucleic acid molecule encoding one of the aforementioned peptides, a CTLA4 agonist or antagonist, a peptidomimetic of a CTLA4 agonist or antagonist, a CTLA4 peptidomimetic, or other small molecule identified using the screening methods described herein. In a preferred embodiment, an agent that modulates CTLA4 binding to PP2AA binds to the lysine-rich motif of CTLA4. In another preferred embodiment, an agent that modulates CTLA4 binding to PP2AA binds to PP2AA and inhibits PP2AA from binding to CTLA4.

These modulatory methods can be performed in vitro (e.g., by contacting the cell with the agent) or, alternatively, in vivo (e.g., by administering the agent to a subject). As such, the present invention provides methods of treating an individual afflicted with a condition or disorder that would benefit from up- or down-modulation of a CTLA4 polypeptide, e.g., a disorder characterized by an unwanted, insufficient, or aberrant immune response. In one embodiment, the method involves administering an agent (e.g., an agent identified by a screening assay described herein), or combination of agents that modulates (e.g., upregulates or downregulates) CTLA4 activity by modulating CTLA4 binding to PP2AA (e.g., via the lysine-rich motif of CTLA4).

Stimulation of CTLA4 activity is desirable in situations in which CTLA4 is abnormally downregulated and/or in which increased CTLA4 activity is likely to have a beneficial effect, for example in a situation of an excessive or unwanted immune response. Such situations include conditions, disorders, or diseases such as an autoimmune disorder (e.g., rheumatoid arthritis, myasthenia gravis, autoimmune thyroiditis, systemic lupus erythematosus, type I diabetes mellitus, Grave's disease, or multiple sclerosis), a transplant (e.g., a bone marrow transplant, a stem cell transplant, a heart transplant, a lung transplant, a liver transplant, a kidney transplant, a cornea transplant, or a skin transplant), graft versus host disease (GVHD), an allergy, or in inflammatory disorder. Likewise, inhibition of CTLA4 activity is desirable in situations in which CTLA4 is abnormally upregulated and/or in which decreased CTLA4 activity is likely to have a beneficial effect.

Exemplary agents for use in upmodulating CTLA4 (i.e., CTLA4 agonists) include, e.g., nucleic acid molecules encoding CTLA4 polypeptides, CTLA4 peptides, and compounds that inhibit the interaction of CTLA4 with PP2AA (e.g., compounds that bind a lysine rich motif and compounds identified in the subject screening assays.

Exemplary agents for use in downmodulating CTLA4 (i.e., CTLA4 antagonists) include agents that stimulate the interaction between CTLA4 and PP2AA (e.g., via the lysine rich motif) in an immune cell (e.g., compounds identified in the subject screening assays).

In one aspect, the invention provides a method for preventing in a subject, a disease or condition associated with an aberrant or unwanted immune response, by administering to the subject an agent which modulates the interaction of CTLA4 and PP2AA. Subjects at risk for a disease which is caused or contributed to by aberrant or unwanted immune response can be identified by, for example, any or a combination of diagnostic or prognostic assays known in the art. Administration of a prophylactic agent can occur prior to the manifestation of symptoms characteristic of the aberrant immune response, such that a disease or disorder is prevented or, alternatively, delayed in its progression. As is true for therapeutic methods, depending on the type of immune response aberrancy, and whether it is desirable to up or downmoduate CTLA4 activity, for example, a CTLA4 antagonist or CTLA4 agonist agent can be used for treating the subject. The appropriate agent can be determined based on screening assays described herein. In an exemplary embodiment, the agent may be a peptide comprising the amino acid sequence SKMLKKRSP (SEQ ID NO:1), a peptide that binds to PP2AA, a peptide that binds to CTLA4, a CTLA4 cytoplasmic domain, a peptide comprising residues 392-589 of PP2A regulatory subunit A, and a small molecule.

Another aspect of the invention pertains to methods of modulating CTLA4 interaction with PP2AA for therapeutic purposes. The interaction between CTLA4 and PP2AA (e.g., via the lysine-rich motif) of CTLA4 can be modulated in order to modulate the immune response. Because CTLA4 downregulates immune responses, and binding of PP2AA to CTLA4 (e.g., via the lysine-rich motif) inhibits CTLA4 activity, inhibition of PP2AA binding to CTLA4 results in upregulation of CTLA4 activity, and therefore, downregulation of immune responses, whereas downregulation of CTLA4 activity results in upregulation of immune responses.

1. Downregulation of Immune Responses

There are numerous embodiments of the invention for upregulating the inhibitory function of a CTLA4 polypeptide by inhibiting the interaction of CTLA4 and PP2AA (e.g., via the lysine rich motif of CTLA4) to thereby downregulate immune responses. Downregulation can be in the form of inhibiting or blocking an immune response already in progress, or may involve preventing the induction of an immune response. The functions of activated immune cells can be inhibited by downregulating immune cell responses or by inducing specific anergy in immune cells, or both.

For example, CTLA4 interaction with PP2AA can be inhibited by contacting a cell which expresses CTLA4 and PP2AA with an agent that inhibits their interaction. Such an agent can be a compound identified by the screening assays described herein. In another embodiment, the agent is a peptide. In a preferred embodiment, the agent can interact with the lysine rich motif of CTLA4 to interfere with the CTLA4-PP2AA interaction. It will be appreciated that preferred agents for use in the methods of the invention will interfere with the CTLA4-PP2AA interaction but will not otherwise modulate other PP2A activities and/or cellular processes, as described herein.

An immune response can be further inhibited by the use of an additional agent that can thereby downmodulate the immune response, as described further herein.

Agents that promote a CTLA4 activity by inhibiting the interaction of CTLA4 with PP2AA (e.g., small molecules or peptides) can be identified by their ability to inhibit immune cell proliferation and/or effector function, or to induce anergy when added to an in vitro assay. For example, cells can be cultured in the presence of an agent that stimulates signal transduction via an activating receptor (e.g., via a TCR). A number of art-recognized readouts of cell activation can be employed to measure, e.g., cell proliferation or effector function (e.g., cytokine production or phagocytosis) in the presence of the activating agent. The ability of a test agent to block this activation can be readily determined by measuring the ability of the agent to effect a decrease in proliferation or effector function being measured.

In one embodiment of the invention, tolerance is induced against specific antigens by co-administering an antigen with a CTLA4 agonist. For example, tolerance can be induced to specific polypeptides. In one embodiment, immune responses to allergens or foreign polypeptides to which an immune response is undesirable can be inhibited. For example, subjects that receive Factor VIII frequently generate antibodies against this clotting factor. Co-administration of an agent that stimulates CTLA4 activity and/or inhibits its interaction with PP2AA, with recombinant factor VIII (or physically linking CTLA4 to Factor VIII, e.g., by cross-linking) can result in immune response downmodulation.

In another embodiment, immune responses can be downregulated in a subject by removing T cells from the patient, contacting the T cells in vitro with an agent (e.g., a small molecule) that upregulates CTLA4 activity by inhibiting CTLA4-PP2AA interaction, and reintroducing the in vitro-stimulated immune cells into the patient. In another embodiment, a method of downregulating immune responses involves transfecting them with a nucleic acid molecule encoding a CTLA4 molecule with a mutated lysine rich motif or a peptide that inhibits CTLA4-PP2AA interaction (e.g., a peptide comprising a lysine rich motif), such that the cells express the CTLA4 molecule (e.g., in the cell membrane) or the peptide (e.g., in the cytoplasm), and reintroducing the transfected cells into the patient. The ability of the transfected cells to be activated may then be reduced.

In another example, portions of a CTLA4 agonist polypeptide can be linked to a toxin to make a cytotoxic agent capable of triggering the destruction of cells to which it binds.

Downregulating immune responses by activating CTLA4 activity by inhibiting the CTLA4-PP2AA interaction (and thus stimulating the negative signaling function of CTLA4) is useful in downmodulating the immune response, e.g., in situations of tissue, skin and organ transplantation, in graft-versus-host disease (GVHD), or allergies, or in autoimmune diseases such as systemic lupus erythematosus and multiple sclerosis. For example, blockage of immune cell function results in reduced tissue destruction in tissue transplantation. Typically, in tissue transplants, rejection of the transplant is initiated through its recognition as foreign by immune cells, followed by an immune reaction that destroys the transplant. The administration of a molecule which promotes the activity of CTLA4 by inhibiting the interaction of CTLA4 with PP2AA in immune cells (such as a CTLA4 or PP2AA peptide or a small molecule) alone or in conjunction with another downmodulatory agent prior to or at the time of transplantation can inhibit the generation of a costimulatory signal. Moreover, promotion of CTLA4 activity by inhibition of CTLA4-PP2AA interaction (and thus, promotion of a CTLA4 inhibitory signal) may also be sufficient to anergize the immune cells, thereby inducing tolerance in a subject. Induction of long-term tolerance by promoting a CTLA4 mediated inhibitory signal may avoid the necessity of repeated administration of these activating reagents.

To achieve sufficient immunosuppression or tolerance in a subject, it may also be desirable to block the costimulatory function of other molecules. For example, it may be desirable to block the function of B7-1 and B7-2 by administering a soluble form of a combination of peptides having an activity of each of these antigens or blocking antibodies against these antigens (separately or together in a single composition) prior to or at the time of transplantation. Alternatively, it may be desirable to promote inhibitory activity of CTLA4 and inhibit a costimulatory activity of B7-1 and/or B7-2. Other downmodulatory agents that can be used in connection with the downmodulatory methods of the invention include, for example, agents that transmit an inhibitory signal via CTLA4, antibodies that activate an inhibitory signal via CTLA4, blocking antibodies against other immune cell markers, or soluble forms of other receptor ligand pairs (e.g., agents that disrupt the interaction between CD40 and CD40 ligand (e.g., anti CD40 ligand antibodies)), antibodies against cytokines, or immunosuppressive drugs.

Examples of other immunomodulating agents include antibodies that block a costimulatory signal, (e.g., against CD28 or ICOS), antibodies that activate an inhibitory signal via CTLA4, and/or antibodies against other immune cell markers (e.g., against CD40, CD40 ligand, or cytokines), fusion proteins (e.g., CTLA4-Fc or PP2AA-Fc), and immunosuppressive drugs (e.g., rapamycin, cyclosporine A, or FK506).

For example, activating CTLA4 activity by inhibiting the interaction of CTLA4 and PP2AA may also be useful in treating autoimmune disease. Many autoimmune disorders are the result of inappropriate activation of immune cells that are reactive against self tissue and which promote the production of cytokines and autoantibodies involved in the pathology of the diseases. Preventing the activation of autoreactive immune cells may reduce or eliminate disease symptoms. Administration of agents that promote activity of CTLA4 by inhibiting CTLA4 interaction with PP2AA may induce antigen-specific tolerance of autoreactive immune cells which could lead to long-term relief from the disease. Additionally, co-administration of agents which block costimulation of immune cells by disrupting receptor-ligand interactions of B7 molecules with costimulatory receptors may be useful in inhibiting immune cell activation to prevent production of autoantibodies or cytokines which may be involved in the disease process. The efficacy of reagents in preventing or alleviating autoimmune disorders can be determined using a number of well-characterized animal models of human autoimmune diseases. Examples include murine experimental autoimmune encephalitis, systemic lupus erythematosus in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Inhibition of immune cell activation is useful therapeutically in the treatment of allergies and allergic reactions, e.g., by inhibiting IgE production. An agent that promotes CTLA4 activity by inhibiting CTLA4 interaction with PP2AA can be administered to an allergic subject to inhibit immune cell-mediated allergic responses in the subject. Stimulation of CTLA4 activity by inhibition of CTLA4 interaction with PP2AA can be accompanied by exposure to allergen in conjunction with appropriate MHC molecules. Allergic reactions can be systemic or local in nature, depending on the route of entry of the allergen and the pattern of deposition of IgE on mast cells or basophils. Thus, immune cell-mediated allergic responses can be inhibited locally or systemically by administration of an agent that promotes CTLA4 activity by inhibition of CTLA4-PP2AA interaction.

Downregulation of immune cell activation through stimulation of CTLA4 activity by inhibition of CTLA4-PP2AA interaction may also be important therapeutically in pathogenic infections of immune cells (e.g., by viruses or bacteria). For example, in the acquired immune deficiency syndrome (AIDS), viral replication is stimulated by immune cell activation. Stimulation of CTLA4 activity by inhibition of CTLA4-PP2AA interaction may result in inhibition of viral replication and thereby ameliorate the course of AIDS.

Downregulation of immune cell activation via stimulation of CTLA4 activity by inhibition of CTLA4-PP2AA interaction may also be useful in treating inflammatory disorders and in promoting the maintenance of pregnancy when there exists a risk of immune-mediated spontaneous abortion.

2. Upregulation of Immune Responses

Inhibition of CTLA4 activity by enhancing CTLA4 interaction with PP2AA as a means of upregulating immune responses is also useful in therapy. Upregulation of immune responses can be in the form of enhancing an existing immune response or eliciting an initial immune response. For example, enhancing an immune response through inhibition of CTLA4 activity by enhancing CTLA4-PP2AA interaction is useful in cases of infections with microbes, e.g., bacteria, viruses, or parasites. For example, in one embodiment, an agent that inhibits CTLA4 activity by enhancing CTLA4-PP2AA interaction, e.g., a small molecule or a peptide that strengthens the CTLA4-PP2AA interaction, is therapeutically useful in situations where upregulation of antibody and cell-mediated responses, resulting in more rapid or thorough clearance of a virus, would be beneficial. These conditions include viral skin diseases such as Herpes or shingles, in which case such an agent can be delivered topically to the skin. In addition, systemic viral diseases such as influenza, the common cold, and encephalitis might be alleviated by the administration of such agents systemically. In certain instances, it may be desirable to further administer other agents that upregulate immune responses, for example, forms of B7 family members that transduce signals via costimulatory receptors, in order further augment the immune response.

Alternatively, immune responses can be enhanced in an infected patient by removing immune cells from the patient, contacting immune cells in vitro with an agent (e.g., a small molecule) that inhibits the CTLA4 activity by upregulating the interaction between CTLA4 and PP2AA, and reintroducing the in vitro-stimulated immune cells into the patient. In another embodiment, a method of enhancing immune responses involves isolating infected cells from a patient, e.g., virally infected cells, transfecting them with a nucleic acid molecule encoding a form of CTLA4 that binds PP2AA more strongly than the wild type CTLA4 (e.g., a form a CTLA4 with more than one lysine rich motif), such that the cells express all or a portion of the CTLA4 molecule on their surface, and reintroducing the transfected cells into the patient. The transfected cells may be capable of preventing an inhibitory signal to, and thereby activating, immune cells in vivo.

A agent that inhibits CTLA4 activity or enhances CTLA4 interaction with PP2AA can be used prophylactically in therapy against various polypeptides, e.g., polypeptides derived from pathogens for vaccination. Immunity against a pathogen, e.g., a virus, can be induced by vaccinating with a viral polypeptide along with an agent that inhibits CTLA4 activity by promoting CTLA4-PP2AA interaction. Nucleic acid vaccines can be administered by a variety of means, for example, by injection (e.g., intramuscular, intradermal, or the biolistic injection of DNA-coated gold particles into the epidermis with a gene gun that uses a particle accelerator or a compressed gas to inject the particles into the skin (Haynes et al. (1996) J. Biotechnol. 44:37)). Alternatively, nucleic acid vaccines can be administered by non-invasive means. For example, pure or lipid-formulated DNA can be delivered to the respiratory system or targeted elsewhere, e.g., Peyers patches by oral delivery of DNA (Schubbert (1997) Proc. Natl. Acad. Sci. USA 94:961). Attenuated microorganisms can be used for delivery to mucosal surfaces (Sizemore et al. (1995) Science 270:29).

In one embodiment, an agent which inhibits CTLA4 activity by enhancing the interaction between CTLA4 and PP2AA can be administered with class I MHC polypeptides by, for example, a cell transfected to coexpress a CTLA4 polypeptide or blocking antibody and MHC class I α chain polypeptide and β₂ microglobulin to result in activation of T cells and provide immunity from infection. For example, viral pathogens for which vaccines are useful include: hepatitis B, hepatitis C, Epstein-Barr virus, cytomegalovirus, HIV-1, HIV-2, tuberculosis, malaria and schistosomiasis.

Stimulation of an immune response to tumor cells can also be achieved by inhibiting CTLA4 activity by enhancing the interaction between CTLA4 and PP2AA by treating a patient with an agent that enhances CTLA4-PP2AA interaction. Preferred examples of such agents include, e.g., and compounds identified in the subject screening assays and peptides, e.g., a CTLA4 molecule with more than one lysine rich motif.

In another embodiment, the immune response can be stimulated by the inhibition of CTLA4 activity by enhancing CTLA4-PP2AA interaction such that preexisting tolerance is overcome. For example, immune responses against antigens to which a subject cannot mount a significant immune response, e.g., tumor-specific antigens, can be induced by administering an agent that inhibits the activity of CTLA4 activity by enhancing the interaction of CTLA4 and PP2AA. Other CTLA4 antagonists can be used as adjuvants to boost responses to foreign antigens in the process of active immunization.

In one embodiment, immune cells are obtained from a subject and cultured ex vivo in the presence of an agent that that inhibits CTLA4 activity by enhancing CTLA4-PP2AA interaction to expand the population of immune cells. In a further embodiment the immune cells are then administered to a subject. Immune cells can be stimulated to proliferate in vitro by, for example, providing the immune cells with a primary activation signal and a costimulatory signal, as is known in the art. Various forms of CTLA4 polypeptides or agents that inhibit CTLA4 activity by enhancing CTLA4-PP2AA interaction can also be used to costimulate proliferation of immune cells. In one embodiment immune cells are cultured ex vivo according to the method described in PCT Application No. WO 94/29436. The agent can be soluble, attached to a cell membrane or attached to a solid surface, such as a bead.

In an additional embodiment, in performing any of the methods described herein, it is within the scope of the invention to upregulate an immune response by administering one or more additional agents. For example, the use of other agents known to stimulate the immune response, such as cytokines, adjuvants, or stimulatory forms of costimulatory molecules or their ligands can be used in conjunction with an agent that inhibits CTLA4 activity by enhancing the CTLA4-PP2AA interaction.

IV. Pharmaceutical Compositions

The CTLA4 nucleic acid molecules, PP2AA nucleic acid molecules, CTLA4 proteins or portions thereof, PP2AA proteins or portions thereof, and modulators of CTLA4/PP2AA interaction (also referred to herein as “active compounds”) used in the methods of the invention can be incorporated into pharmaceutical compositions suitable for administration to a subject, e.g., a human. Such compositions typically comprise the nucleic acid molecule, protein, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a small molecule, nucleic acid molecule, or peptide) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

Viral vectors include, for example, recombinant retroviruses, adenovirus, adeno-associated virus, and herpes simplex virus-1. Retrovirus vectors and adeno-associated virus vectors are generally understood to be the recombinant gene delivery system of choice for the transfer of exogenous genes in vivo, particularly into humans. Alternatively they can be used for introducing exogenous genes ex vivo into T cells in culture. These vectors provide efficient delivery of genes into T cells, and the transferred nucleic acids are stably integrated into the chromosomal DNA of the host cell.

A major prerequisite for the use of viruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) is replaced by a gene of interest rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm.

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) Proc. Natl. Acad. Sci. USA 86:9079-9083; Julan et al. (1992) J. Gen. Virol. 73:3251-3255; and Goud et al. (1983) Virology 163:251-254); or coupling cell surface receptor ligands to the viral env proteins (Neda et al. (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). Thus, in a specific embodiment of the invention, viral particles containing a nucleic acid molecule containing a gene of interest operably linked to appropriate regulatory elements, are modified for example according to the methods described above, such that they can specifically target subsets of liver cells. For example, the viral particle can be coated with antibodies to surface molecule that are specific to certain types of liver cells. This method is particularly useful when only specific subsets of liver cells are desired to be transfected.

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See for example Berkner et al. (1988) Biotechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells. Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al. (1979) Cell 16:683; Berkner et al., supra; and Graham et al. in Methods in Molecular Biology, E. J. Murray, Ed. (Humana, Clifton, N.J., 1991) vol. 7. pp. 109-127). Expression of the gene of interest comprised in the nucleic acid molecule can be under control of, for example, the E1A promoter, the major late promoter (MLP) and associated leader sequences, the E3 promoter, or exogenously added promoter sequences.

Yet another viral vector system useful for delivery of a nucleic acid molecule comprising a gene of interest is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics Microbiol. Immunol. (1992) 158:97-129). Adeno-associated viruses exhibit a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as few as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into T cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790). Other viral vector systems that may have application in gene therapy have been derived from herpes virus, vaccinia virus, and several RNA viruses.

Other methods relating to the use of viral vectors in gene therapy can be found in, e.g., Kay, M. A. (1997) Chest 111(6 Supp.):138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11:179-86; Thule, P.M. and Liu, J. M. (2000) Gene Ther. 7:1744-52; Yang, N. S. (1992) Crit. Rev. Biotechnol. 12:335-56; Alt, M. (1995) J. Hepatol. 23:746-58; Brody, S. L. and Crystal, R. G. (1994) Ann. N. Y Acad. Sci. 716:90-101; Strayer, D. S. (1999) Expert Opin. Invetig. Drugs 8:2159-2172; Smith-Arica, J. R. and Bartlett, J. S. (2001) Curr. Cardiol Rep. 3:43-49; and Lee, H. C. et al. (2000) Nature 408:483-8.

The pharmaceutical compositions can be included in a container, pack, or dispenser together with instructions for administration.

This invention is further illustrated by the following examples which should not be construed as limiting. The contents of all references, patents, and published patent applications cited throughout this application, as well as the figures and the sequence listing, are hereby incorporated by reference.

### EXAMPLES

Materials and Methods

Cells

The panel of Jurkat T cells transfected with a regulatable, doxycycline sensitive CTLA4 cDNA has been previously described (Carreno, B. M. et al. (2000) J. Immunol. 165:1352-1356; Baroja, M. L. et al. (2000) J. Immunol. 164:49-55). A luciferase reporter cDNA under the control of the interleukin-2 promoter and enhancer elements was transfected in these cells, and clones isolated after limiting dilution were used for these experiments. A 0.45 lymphoblastoid B cell line that expresses HLA-DR1 and B7.1 was also used. Both cell lines were cultured under standard conditions.

Plasmid Construction

The cytoplasmic regions of mouse CTLA4 ,CD28, and ICOS were generated by polymerase chain reaction (PCR). The cDNA encoding the murine CTLA4 cytoplasmic domain was inserted into the EcoRI site of bait vector PEG202 (Origene Technologies, Inc., Rockville, Md.) to yield a Lex-A DNA binding fusion plasmid. Similarly, cytoplasmic regions of murine CD28 and ICOS were subcloned into the EcoR1 site of bait vector PEG202.

The 1 kb SalI fragment of clone 54 or the 1.7 kb SalI fragment of full length murine PP2AA were inserted into the SalI site of vector pCMV-myc (Clontech), which contains an oligonucleotide encoding the myc peptide inserted into the 5′ end of the multiple cloning site of the mammalian expression vector pCMV. Full length CTLA4 was expressed as an HA-tagged fusion protein by inserting the CTLA4 cDNA into the EcoRI/Xhol sites of pCMV-HA (Clontech). Similarly, full length CD28 and ICOS were cloned into the EcoRI/SalI sites of pCMV-HA.

Yeast Two Hybrid System

A yeast two hybrid screen of a murine Th1 T cell library was performed by cotransfecting the bait CTLA4-PEG202 into the yeast strain EGY4-8 (Origene Technologies) along with a murine Th1 T cell library constructed in the B42 activation domain of pJG4-5 (Origene Technologies, Inc.), using the Duplex A yeast two hybrid system (Origene Technologies, Inc.). Positive interaction was confirmed by expression of both the Leu2 and LacZ genes, thereby confirming the ability of positive clones to grow on media lacking leucine and to turn blue on media containing X-Gal. Screening was performed on 2 million transformants. To establish the specificity of the interaction, plasmids containing clones 54 and 48, identified as molecules interacting with the cytoplasmic domain of CTLA4 in yeast, were reisolated from the library plasmid, pJG4-5. The was then retransformed into EGY 4-8 yeast along with the cytoplasmic domain of CTLA4, CD28, or ICOS. Positive interaction was scored by the expression of Leu2 or LacZ on media containing Leu/Gal or Gal/X-Gal.

Protein Interactions

The plasmid vectors encoding the myc-tagged PP2AA (residues 392-589) or full length PP2AA and HA-tagged CD28, CTLA4 or vector alone were cotransfected into human 293 cells by using Lipofectamine, according to the manufacturer's instructions. Transfected cells were harvested and lysed at 4° C. in 1% NP40 lysis buffer. Cell lysates were precleared and immunoprecipitated using protein G beads coated with anti-HA antibodies or anti-myc antibodies. After an overnight incubation at 4° C., the immunoprecipitates were washed using 1% NP40 lysis buffer. Bound proteins were eluted by boiling in SDS sample buffer, separated by SDS/15% PAGE, and transferred onto PVDF membranes. Membranes were blocked with 3% BSA in PBS and then incubated with anti-mPP2AA or anti-myc antibodies for detecting PP2AA. For the detection of CTLA4 and CD28, PVDF membranes were blotted with anti-CD28 and anti-CTLA4 antibodies. Subsequently, the membranes were incubated with horseradish peroxidase (HRP) conjugated secondary antibodies, before visualization using chemiluminescence reagents.

Biochemistry

Jurkat T cells (cell number normalized for protein content) were cultured overnight in the absence or presence of doxycycline and were stimulated for 10 minutes with 0.45 lymphoblastoid B cells (5:1 ratio) preincubated with SEE (1 ng/ml) (Toxin Technology Inc., Sarasota, Fla.) for 40 minutes at 37° C. Cells were subsequently lysed in standard lysis buffer containing Triton X-100 (1%). Lysates were precleared with protein G agarose beads (Roche, Laval, Quebec), followed by immunoprecipitation with DSP-crosslinked antibodies on protein G agarose beads, and Western blotted as previously described (Baroja, M. L. et al. (2000) supra; Madrenas, J. et al. (1997) J. Exp. Med. 185:219-229; Chau, L. A. et al. (1998) J. Exp. Med. 187:1699-1709).

Reagents

Antibodies used in the examples were: a goat polyclonal antiserum against the PP2AAα regulatory subunit (Santa Cruz Biotechnology, Santa Cruz, Calif.), a mouse monoclonal antibody against the PP2AC catalytic subunit (either from Santa Cruz Biotechnology or from Upstate Biotechnology, Lake Placid, N.Y.), a mouse monoclonal antibody against phosphotyrosine, a mouse monoclonal antibody against human CTLA4. 11, a chimeric B7.2-hIgG1 molecule (Genetics Institute, Inc., Cambridge, Mass.), and a goat polyclonal antiserum against a peptide from the extracellular portion of human CD28 (Santa Cruz Biotechnology, Santa Cruz, Calif.).

Luciferase Assay

Doxycycline treated Jurkat T cells (0.25×10⁶ cells/group) were stimulated for 4 hours with 0.45 lymphoblastoid B cells (ratio 2:1) preincubated overnight with different concentrations of SEE. Luciferase assays were performed using the Promega Luciferase Assay System (Promega, Madison, Wis.).

Flow Cytometry

Jurkat cells (1×10⁶ cells/group) were cultured overnight in the absence or presence of doxycycline to induce CTLA4 expression. Subsequently, the cells were washed and stained for CTLA4 expression using a PE-labeled monoclonal antibody against human CTLA4 (Pharmingen). Cells were examined by flow cytometry using a FACScan Flow Cytometer (Becton Dickinson, Mountain View, Calif.). Statistical analyses were performed with CELLQuest computer software.

### Example 1

### Interaction of CTLA4 and PP2AA (392-589) In Yeast

To gain an understanding of the molecular mechanisms involved in CTLA4 mediated T cell down-regulation, a yeast-two hybrid screen was used to identify putative proteins interacting with the cytoplasmic domain of CTLA4. Since it was more likely that such proteins would be expressed in an activated T cell, an activated Th1 T cell library was screened using the cytoplasmic domain of mouse CTLA4 fused to the DNA binding domain of Lex-A as bait. Of the 2 million transformants screened, 2 clones interacted specifically when tested for nutritional selection and β-galactosidase activity. Both clones were identified as containing a cDNA insert spanning amino acids Ala³⁹²-Asp⁵⁸⁹ of the carboxy-terminal end of PP2AA.

To verify the specificity of this interaction, the ability of the mouse 392-589 domain of PP2AA (mPP2AA(392-589)) to interact with CD28 was determined. CD28 is a closely related molecule which shares extensive structural and sequence homologies with CTLA4. As an additional control, the cytoplasmic tail of the newly discovered CD28 family member ICOS was also used. cDNAs encoding the cytoplasmic domains of CD28 and ICOS were cloned in the bait vector and used to retransform yeast cells along with mPP2AA(392-589). When assessed for growth on nutritionally selective media and β-galactosidase activity, it was found that mPP2AA(392-589) did not interact with CD28 or ICOS, demonstrating that the mPP2AA(392-589) contained an interacting motif specific for the cytoplasmic domain of mouse CTLA4. To confirm this observation in a mammalian cell system, clone 54, representing mPP2AA(392-589), was expressed as a myc-tagged protein of 23 kD in H293K cells cotransfected with HA tagged full length CTLA4 or CD28 molecules. When antibodies against HA were used to immunoprecipitate HA-CD28 or HA-CTLA4, only CTLA4, but not CD28, was found to associate with mPP2AA(392-589). The absence of interaction between mPP2AA(392-589) and CD28 even in a H293K expression system verified the findings in the yeast system. Additionally, it suggested that the domain of mPP2AA encompassing amino acids 392-589 probably contains anchor residues that mediate binding to CTLA4 but not its close homolog CD28.

### Example 2

### Interaction of CTLA2 with Full Length PP2AA

In order to extend this observation to the ubiquitously expressed, full length PP2AA, the full length PP2AA was amplified from activated murine spleen cells. Recombinant mPP2AA migrated as a 61 kD protein when expressed in H293K cells. To test for interaction of full length PP2AA with CTLA4, PP2AA was expressed as a myc-tagged protein and cotransfected with either HA-CTLA4, HA-CD28, or HA-vector alone. Upon immunoprecipitating the lysates of H293K cells with anti-HA antibody, both PP2AA-CTLA4 and PP2AA-CD28 immune complexes could be detected by Western blotting. This was in marked contrast to the results observed with mPP2AA(392-589) domain in both yeast and H293K cells. Together, the yeast two hybrid screen and the co-immunoprecipitation data in the H293K system indicated that the full-length mPP2AA can interact with both CD28 and CTLA4. However, these molecules associate with PP2AA utilizing distinct domains for interaction. Specifically, the domain containing residues 392-589 binds exclusively to CTLA4, but residues 1-392 either alone or together with other residues associate with CD28.

### Exampe 3

### Interaction of CTLA4 with PP2AA in Transfected Inducible Jurkat Cells

A well characterized system in which Jurkat cells are induced to express transfected CTLA4 upon exposure to doxycycline (Carreno, B. M. et al. (2000) J. Immunol. 165:1352-1356; Baroja, M. L. et al. (2000) J. Immunol. 164:49-55) was used to investigate the functional relevance of PP2AA in CTLA function. The Jurkat cells were first examined for expression of PP2AA. Resting, non-induced Jurkat cells expressed abundant PP2AA that migrated as a 65 kD band on a Western blot. The level of endogenous PP2AA expression remained constant even after doxycycline mediated induction of CTLA4. Thus, the levels of endogenous PP2AA are not affected by doxycycline treatment of Jurkat cells.

In transfected Jurkat cells, it was found that immunoprecipitation of the 65 kD regulatory subunit of PP2A coprecipitated a band with a size and blotting reactivity comparable to that of CTLA4. Since Jurkat cells express a TCR Vα1/Vβ8.1 antigen receptor complex, the effects of TCR or TCR-CTLA4 coligation were tested using a system in which the superantigen staphylococcal enterotoxin E (SEE) is presented by HLA DRI -expressing, B7.1⁺ antigen presenting cells (APCs) (Makida, R. et al. (1996) Mol. Immunol. 33:891-900). CTLA4 transfected Jurkat T cells (30×10⁶ cells/group) were stimulated with antigen presenting cells (0.45 lymphoblastoid B cells) and SEE (1 ng/ml) for 10 minutes, with or without prior induction of CTLA4 expression by overnight incubation with doxycycline (5 μg/ml). Subsequently, cell lysates were prepared and used for immunoprecipitation of PP2AA, followed by immunoblotting for CTLA4 or PP2AA. Whole cell lysates from the same samples were used for direct immunoblotting for CTLA4 to confirm induction of CTLA4 expression. A non-lymphoid cell line (H293K) and 0.45 cells were used as controls.

Upon stimulation of doxycycline induced Jurkat cells with SEE and APCs to induce TCR-CTLA4 coligation, there was a 53% decrease in PP2AA:CTLA4 association as compared to non-stimulated cells. In addition, since parental Jurkat cells (E6.1 cells) only express CD28 but not CTLA4, these cells were utilized to establish the association of PP2AA with CD28 using a B7-Ig fusion protein. Western blotting with anti-PP2AA antibody after immunoprecipitation with B7.21g revealed that PP2A also bound to CD28. These data confirm the findings obtained in the H293K system in that PP2AA associated with CD28 as well as CTLA4 (see above).

### Example 4

### TCR Ligation Induces Tyrosine Phosphorylation of PP2AA

Previous reports have implicated phosphorylation of the catalytic subunit of PP2A by several kinases including p561ck in the inactivation of PP2A (Chen, J. et al. (1992) Science 257:1261-1264). This example describes the determination of whether TCR ligation could result in the tyrosine phosphorylation of PP2AA. TCR ligation by SEE and APCs caused an increase in tyrosine phosphorylation of the regulatory subunit of PP2A in a time dependent fashion. Furthermore, co-ligation of the TCR and CTLA4 with SEE and APCs resulted in a time dependent decrease in the levels of PP2AA associated with CTLA4, while the total levels of PP2AA and PP2AC remained constant.

### Example 5

### Identification of a Lysine Rich Motif Which Mediates the Interaction of CTLA4 AND PP2AA

Various studies have documented the importance of the PP2A holoenzyme as both a negative and positive regulator of cell growth and cell cycle progression proteins (Sontag, E. et al. (1993) Cell 75:887-897; Heriche, J. K. et al. (1997) Science 276:952-955; Millward, T. A. et al. (1999) Trends. Biochem. Sci. 24:186-191). Considering the fact that both PP2AA and CTLA4 exists in resting T cells, as shown herein, the association of PP2AA and CTLA4 in T cells may be a mechanism by which the phosphatase prevents the inhibitory function of CTLA4 prior to TCR-CTLA4 coligation. Additionally, activation and subsequent coligation of CTLA4 and TCR could result in the tyrosine phosphorylation of both CTLA4 and PP2AA resulting in retention of CTLA4 on the cell surface and the dissociation of CTLA4 from PP2AA, respectively. Reversible phosphorylation of PP2A and its association with various intracellular molecules that regulate cell cycle progression has been previously reported (Sontag et al. (1993) supra; Xu, Z. and Williams, B. R. (2000) Mol. Cell Biol. 20:5285-5299). Dissociation from PP2AA could then result in the restoration of CTLA4 functional activity. Accordingly, based on the data reported herein, it was hypothesized that a mutant CTLA4 incapable of binding to PP2AA would be a better inhibitor of T cell function than the wild type molecule.

It has been reported that the sequence HENRKL (SEQ ID NO:10) in the SV40 small T antigen and in the kinase domain of Casein kinase 2α is the sequence required for binding of these proteins to the PP2A core enzyme (Sontag et al. (1993) supra; Heriche et al. (1997) supra). Based on this data, the presence of a similar sequence in the cytoplasmic tails of CTLA4 and in those proteins known to form stable complexes with PP2A (see Millward, T. A. et al. (1999) Trends Biochem. Sci. 24:186-191) was investigated. It was found that the cytoplasmic tail of CTLA4 contained a K-rich motif, SKMLKKRSP (SEQ ID NO:1) in the juxtamembrane portion of its cytoplasmic tail. Such sequence meets a consensus also found in other PP2A-binding proteins: X-[K/R/H]-X-X-[K/R/H]-K-X-X-X (SEQ ID NO:2 (Sontag, E. et al. (1993) Cell 75:887-897; Heriche, J. K. et al. (1997) Science 276:952-955; Millward et al. (1999) supra), and located within regions identified as important for binding to the regulatory subunit of PP2A (FIG. 1; Campbell, K. S. et al. (1995)J. Virol. 69:3721-3728). As used herein, the letter “X” in the consensus sequence signifies any amino acid residue at the indicated position, the notation [K/R/H] signifies any one of K (lysine), R (arginine), or H (histidine) at the indicated position, and one-letter codes for the amino acid residues are used according the to the IUPAC standard. No similar sequence was found in the cytoplasmic tail of CD28, which explains the differential binding observed for PP2AA between CD28 and CTLA4, as described herein.

### Example 6

### The Lysine Residues in the Lysine Rich Motif are Critical for Binding of CTLA4 to PP2AA

A Jurkat T cell clone that expressed a mutant K-less CTLA4 molecule lacking the three lysine residues in the juxtamembrane region (K152A/K155A/K156A) was generated. Each of the three lysines in the lysine rich motif was mutated to alanine. The cells were stained with an antibody against CTLA4 in the presence of increasing concentrations of doxycycline for 18 hours and examined by flow cytometry. Upon induction with doxycycline, the K-less CTLA4 mutant was found to be expressed at significantly lower levels than wild type CTLA4.

It was determined whether the mutant K-less CTLA4, which lacks potential anchor residues that mediate interaction with PP2AA, is still capable of forming CTLA4-PP2A complexes. Jurkat cell lysates from cells normalized for total levels of CTLA4 were used to immunoprecipitate PP2AA. Unlike the wild type CTLA4, the mutant K-less CTLA4 failed to co-immunoprecipitate with PP2AA. This was not due to the inability or decreased reactivity of anti-CTLA4 antibodies used to detect mutant-K less CTLA4. This finding confirmed that the lysine residues are critical for binding of CTLA4 to PP2AA. Furthermore, it offered us an opportunity to delineate the functional relevance of the CTLA4-PP2A interaction in T cells.

### Example 7

### K-Less CTLA4 Increases Inhibition of T Cell Activation

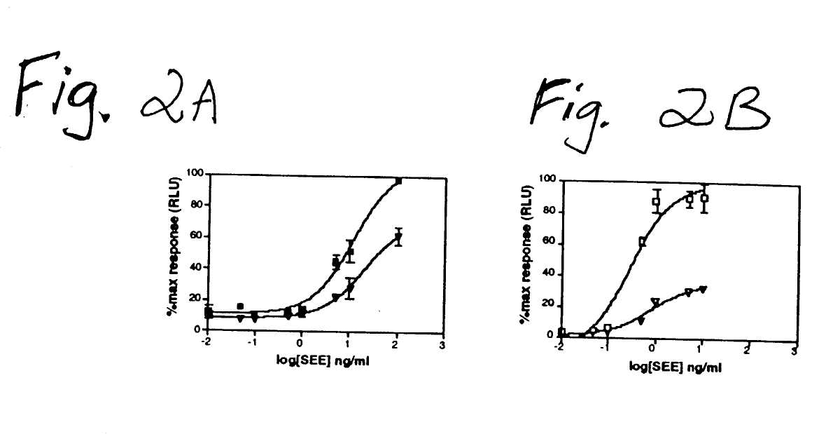

To assess the functional effects of K-less CTLA4 on T cell responses, Jurkat cells expressing wild type CTLA4 or mutant K-less CTLA4 were co-transfected with a luciferase reporter gene under the control of the IL-2 promoter and enhancer elements. Upon stimulation of doxycycline-induced Jurkat cells with SEE and APCs, it was observed that K-less CTLA4 was far more efficient than wild type CTLA4 at inhibiting IL-2 gene transcription (FIGS. 2A and 2B). The enhanced inhibition of the K-less mutant was verified by comparing the percentages of inhibition at maximal RLU response, in order to rule out intrinsic differences between the mutant's ability to transcribe IL-2. Specifically, luciferase activity was inhibited by 70-80% in K-less mutants, compared to 35-55% by the wild type CTLA4 upon TCR-CTLA4 co-ligation. This enhanced inhibition is particularly significant in the context of the much lower surface expression of K-less CTLA4. Furthermore, confocal studies probing for the ability of TCR/CTLA4 receptors to co-cap at the immunological synapse showed that CD3 and CTLA4 receptor reorganization was unaffected in both wild type and K-less Jurkat cells. Thus, the lack of association between PP2AA and K-less CTLA4 correlated with an enhanced inhibition of IL-2 gene transcription by CTLA4.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for modulating an immune response comprising contacting a cell expressing at least one first molecule having a CTLA4 lysine rich motif and at least one second molecule having a PP2AA CTLA4-interacting domain with an agent that modulates the interaction between the first molecule and the second molecule to thereby modulate the immune response.

2. The method of claim 1, wherein the cell is a T cell.

3. The method of claim 2, wherein anergy is induced in the T cell.

4. The method of claim 1, wherein the immune response is downregulated.

5. The method of claim 1, wherein the agent interacts with the lysine rich motif of CTLA4.

6. The method of claim 1, wherein the agent interacts with amino acid residues 392-589 of PP2AA.

7. The method of claim 1, wherein the agent is selected from the group consisting of: a peptide comprising the amino acid sequence SKMLKKRSP (SEQ ID NO:1), a peptide that binds to a PP2AA molecule, a peptide that binds to a CTLA4 molecule, a CTLA4 cytoplasmic domain or a portion thereof, a peptide comprising residues 392-589 of PP2AA, and a small molecule.

8. The method of claim 1, further comprising contacting the cell with at least one additional agent that downregulates an immune response.

9. The method of claim 1, wherein the step of contacting occurs in vivo.

10. The method of claim 1, wherein the step of contacting occurs in vitro.

11. The method of claim 1, wherein the interaction between the first molecule and the second molecule is downregulated.

12. A method for treating a subject having a condition that would benefit from downregulation of an immune response comprising administering an agent that inhibits the interaction between interaction between a first molecule having a CTLA4 lysine rich motif and a second molecule having a PP2AA CTLA4-interacting domain in at least one T cell of the subject such that a condition that would benefit from downregulation of an immune response is treated.

13. The method of claim 12, wherein signaling via a T cell receptor in the at least one T cell of the subject is downregulated.

14. The method of claim 12, wherein anergy is induced in the at least one T cell of the subject.

15. The method of claim 12, wherein the agent interacts with the lysine rich motif of CTLA4.

16. The method of claim 12, wherein the agent interacts with amino acid residues 392-589 of PP2AA.

17. The method of claim 12, wherein the agent is selected from the group consisting of: a peptide comprising the amino acid sequence SKMLKKRSP (SEQ ID NO:1), a peptide that binds to a PP2AA, a peptide that binds to a CTLA4 molecule, a CTLA4 cytoplasmic domain or a portion thereof, a peptide comprising residues 392-589 of PP2AA, and a small molecule.

18. The method of claim 12, further comprising administering to the subject at least one additional agent that downregulates an immune response.

19. The method of claim 12, wherein the interaction between the first molecule and the second molecule is downregulated.

20. The method of claim 12, wherein the condition is selected from the group consisting of: an autoimmune disorder, a transplant, graft versus host disease, an allergy, and an inflammatory disorder.

21. The method of claim 20, wherein the autoimmune disorder is selected from the group consisting of: rheumatoid arthritis, myasthenia gravis, autoimmune thyroiditis, systemic lupus erythematosus, type I diabetes mellitus, Grave's disease, and multiple sclerosis.

22. The method of claim 20, wherein the transplant is selected from the group consisting of: a bone marrow transplant, a stem cell transplant, a heart transplant, a lung transplant, a liver transplant, a kidney transplant, a cornea transplant, or a skin transplant.

23. A method for treating a subject having a condition that would benefit from downregulation of an immune response, comprising:
a) contacting T cells expressing at least one first molecule having a CTLA4 lysine rich motif and at least one second molecule having a PP2AA CTLA4-interacting domain from the subject with an agent that modulates the interaction between the first molecule and the second molecule, and
b) administering the T cells to the subject,
such that a condition that would benefit from downregulation of an immune response is treated.

24. The method of claim 23, wherein signaling via T cell receptors in the T cells from the subject is downregulated.

25. The method of claim 23, wherein anergy is induced in the T cells of the subject.

26. The method of claim 23, wherein the agent interacts with the lysine rich motif of CTLA4.

27. The method of claim 23, wherein the agent interacts with amino acid residues 392-589 of PP2AA.

28. The method of claim 23, wherein the agent is selected from the group consisting of: a peptide comprising the amino acid sequence SKMLKKRSP (SEQ ID NO:1), a peptide that binds to a PP2AA molecule, a peptide that binds to a CTLA4 molecule, a CTLA4 cytoplasmic domain or a portion thereof, a peptide comprising residues 392-589 of PP2AA, and a small molecule.

29. The method of claim 23, further comprising administering to the subject at least one additional agent that downregulates an immune response.

30. The method of claim 23, wherein the interaction between the first molecule and the second molecule is downregulated.

31. The method of claim 23, wherein the condition is selected from the group consisting of: an autoimmune disorder, a transplant, graft versus host disease, an allergy, and an inflammatory disorder.

32. The method of claim 31, wherein the autoimmune disorder is selected from the group consisting of: rheumatoid arthritis, myasthenia gravis, autoimmune thyroiditis, systemic lupus erythematosus, type I diabetes mellitus, Grave's disease, and multiple sclerosis.

33. The method of claim 31, wherein the transplant is selected from the group consisting of: a bone marrow transplant, a stem cell transplant, a heart transplant, a lung transplant, a liver transplant, a kidney transplant, a cornea transplant, or a skin transplant.

34. A method for identifying a compound which modulates the interaction of CTLA4 and PP2AA comprising contacting a cell comprising at least one first molecule having a CTLA4 cytoplasmic domain containing a lysine rich motif and at least one second molecule having a PP2AA CTLA4-interacting domain with a test compound and determining the ability of the test compound to modulate the interaction of the first molecule and the second molecule.

35. The method of claim 34, wherein the first molecule is derived from an exogenous source.

36. The method of claim 34, wherein the second molecule is derived from an exogenous source.

37. The method of claim 34, wherein the second molecule comprises amino acid residues 392-589 of PP2AA.

38. The method of claim 34, wherein the interaction of the first molecule and the second molecule is inhibited.

39. The method of claim 34, wherein the cell is a yeast cell.

40. The method of claim 39, wherein determining the ability of the test compound to modulate the interaction of the first molecule and the second molecule comprises determining the ability of the compound to modulate growth of the yeast cell on nutritionally selective media.

41. The method of claim 39, wherein determining the ability of the test compound to modulate the interaction of the first molecule and the second molecule comprises determining the ability of the compound to modulate expression of a LacZ reporter gene in the yeast cell.

42. The method of claim 34, wherein the cell is a T cell.

43. The method of claim 34, wherein determining the ability of the test compound to modulate the interaction of the first molecule and the second molecule comprises determining the ability of the test compound to modulate the coimmunoprecipitation of the first molecule and the second molecule.

44. The method of claim 42, wherein determining the ability of the test compound to modulate the interaction of the first molecule and the second molecule comprises determining the ability of the test compound to modulate cytokine production by the T cell.

45. The method of claim 44, wherein determining the ability of the test compound to modulate cytokine production by the T cell comprises determining the ability of the compound to modulate the activity of a reporter gene operatively linked to the IL-2 promoter/enhancer region in the T cell.

46. The method of claim 44, wherein determining the ability of the test compound to modulate the interaction of the first molecule and the second molecule comprises determining the ability of the test compound to modulate proliferation of the T cell.

47. A method for identifying a compound which modulates the interaction of a CTLA4 molecule and a PP2AA molecule comprising:
a) contacting, in the presence of the compound, a first molecule comprising at least a portion of the CTLA4 molecule and a second molecule comprising at least a portion of the PP2AA molecule under conditions which allow binding of the first molecule and the second molecule to form a complex; and
b) detecting the formation of a complex of the first molecule and the second molecule in which the ability of the compound to modulate interaction between the first molecule and the second molecule is indicated by a change in complex formation as compared to the amount of complex formed in the absence of the compound.

48. The method of claim 47, wherein the first molecule comprises a CTLA4 cytoplasmic domain.

49. The method of claim 47, wherein the first molecule comprises at least one lysine rich motif.

50. The method of claim 47, wherein the second molecule comprises amino acid residues 392-589 of PP2AA.

51. The method of claim 47, wherein detecting the formation of a complex of the first molecule and the second molecule comprises detecting coimmunoprecipitation of the first molecule and the second molecule.

52. The method of claim 47, wherein the formation of a complex of the first molecule and the second molecule is inhibited by the compound.

53. A method for identifying a compound which modulates the interaction of a molecule comprising at least one CTLA4 lysine rich motif and a PP2AA molecule comprising a PP2AA CTLA4-interacting domain comprising:
a) contacting the molecule comprising at least one CTLA4 lysine rich motif with the compound; and
b) detecting binding of the compound to the CTLA4 lysine rich motif of the molecule,
to thereby identify a compound which modulates the interaction of a molecule comprising at least one CTLA4 lysine rich motif and a PP2AA molecule.

54. The method of claim 53, wherein the molecule comprising at least one CTLA4 lysine rich motif consists of at least one CTLA4 lysine rich motif.


SEQUENCE LISTING (25 sequences)

SEQ ID NO: 1 - protein, 9 aa, Homo sapiens
SKMLKKRSP

SEQ ID NO: 2 - DNA, 672 bp, Homo sapiens
atggcttgccttggatttcagcggcacaaggctcagctgaacctggctgccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgacggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattga

SEQ ID NO: 3 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGAQIYVIDPEPCPDSDFLLWILAAVSSGLFFYSFLLTAVSLSKMLKKRSPLTTGVYVKMPPTEPECEKQFQPYFIPIN

SEQ ID NO: 4 - DNA, 1890 bp, Mus musculus
  misc_feature 1390: n = A,T,C or G
gatcctgttgggttttactctactccctgaggacctcagcacatttgccccccagccatggcttgtcttggactccggaggtacaaagctcaactgcagctgccttctaggacttggccttttgtagccctgctcactcttcttttcatcccagtcttctctgaagccatacaggtgacccaaccttcagtggtgttggctagcagccatggtgtcgccagctttccatgtgaatattcaccatcacacaacactgatgaggtccgggtgactgtgctgcggcagacaaatgaccaaatgactgaggtctgtgccacgacattcacagagaagaatacagtgggcttcctagattaccccttctgcagtggtacctttaatgaaagcagagtgaacctcaccatccaaggactgagagctgttgacacgggactgtacctctgcaaggtggaactcatgtacccaccgccatactttgtgggcatgggcaacgggacgcagatttatgtcattgatccagaaccatgcccggattctgacttcctcctttggatccttgtcgcagttagcttggggttgtttttttacagtttcctggtctctgctgtttctttgagcaagatgctaaagaaaagaagtcctcttacaacaggggtctatgtgaaaatgcccccaacagagccagaatgtgaaaagcaatttcagccttattttattcccatcaactgaaaggccgtttatgaagaagaaggagcatacttcagtctctaaaagctgaggcaatttcaactttccttttctctccagctatttttacctgtttgtatattttaaggagagtatgcctctctttaatagaaagctggagcaaaattccaattaagcatactacaatttaaagctaaggagcagaacagagagctgggatatttctgttgtgtcagaaccattttactaaaagcatcacttggaagcagcataaggatatagcattatggtgtggggtcaagggaacattagggaatggcacagcccaaagaaaggaagggggtgaaggaagagattatattgtacacatcttgtatttacctgagagggggtgaaggaagagattatattgtacacatcttgtatttacctgagagatgtttatgacttaaataatttttaaatttttcatgctgttattttctttaacaatgtataattacacgaaggtttaaacatttattcacagagactatgtgacatagccagtggttccaaaggttgtagtgttccaagatgtatttttaagtaatattgtacatgggtgtttcatgtgctgttgtgtatttgctggtggtttgaatataaacactatgtatcagtgtcgtcccacagtgggtcntggggaggtttggctggggagcttaggacactaatccatcaggttggactcgaggtcctgcaccaactggcttggaaactagatgaggctgtcacagggctcagttgcataaaccgatggtgatggagtgtgggctgggtctttacactcattttattttttgtttctgcttttgttttcttcaatgatttgcaaggaaaccaaaagctggcagtgtttgtatgaacctgacagaacactgtcttcaaggaaatgcctcattcctgagaccagtaggtttgtttttttaggaagttccaatactaggaccccctacaagtactatggctcctcgaaaacacaaagttaatgccacaggaagcagcagatggtaggatgggatgcacaagagttcctgaaaactaacactgttagtgttttttttttaactcaatattttccatgaaaatgcaaccacatgtataatatttttaattaaataaaagtttcttgtgattgtt

SEQ ID NO: 5 - protein, 223 aa, Mus musculus
MACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDFLLWILVAVSLGLFFYSFLVSAVSLSKMLKKRSPLTTGVYVKMPPTEPECEKQFQPYFIPIN

SEQ ID NO: 6 - DNA, 2205 bp, Homo sapiens
gaattccggttctcactcttgacgttgtccagctccagcaccttggcaactcccccagcttggacggccggcccgccgctccatgggggagtcatctgagcacagctgctggccgcagtctgacaggaaagggacggagccaagatggcggcggccgacggcgacgactcgctgtaccccatcgcggtgctcatagacgaactccgcaatgaggacgttcagcttcgcctcaacagcatcaagaagctgtccaccatcgccttggcccttggggttgaaaggacccgaagtgagcttctgcctttccttacagataccatctatgatgaagatgaggtcctcctggccctggcagaacagctgggaaccttcactaccctggtgggaggcccagagtacgtgcactgcctgctgccaccgctggagtcgctggccacagtggaggagacagtggtgcgggacaaggcagtggagtccttacgggccatctcacacgagcactcgccctctgacctggaggcgcactttgtgccgctagtgaagcggctggcgggcggcgactggttcacctcccgcacctcggcctgcggcctcttctccgtctgctacccccgagtgtccagtgctgtgaaggcggaacttcgacagtacttccggaacctgtgctcagatgacacccccatggtgcggcgggccgcagcctccaagctgggggagtttgccaaggtgctggagctggacaacgtcaagagtgagatcatccccatgttctccaacctggcctctgacgagcaggactcggtgcggctgctggcggtggaggcgtgcgtgaacatcgcccagcttctgccccaggaggatctggaggccctggtgatgcccactctgcgccaggccgctgaagacaagtcctgggccgtccgctacatggtggctgacaagttcacagagctccagaaagcagtggggcctgagatcaccaagacagacctggtccctgccttccagaacctgatgaaagactgtgaggccgaggtgagggccgcagcctcccacaaggtcaaagagttctgtgaaaacctctcagctgactgtcgggagaatgtgatcatgtcccagatcttgccctgcatcaaggagctggtgtccgatgccaaccaacatgtcaagtctgccctggcctcagtcatcatgggtctctctcccatcttgggcaaagacaacaccatcgagcacctcttgcccctcttcctggctcagctgaaggatgagtgccctgaggtacggctgaacatcatctctaacctggactgtgtgaacgaggtgattggcatccggcagctgtcccagtccctgctccctgccattgtggagctggctgaggacgccaagtggcgggtgcggctggccatcattgagtacatgcccctcctggctggacagctgggagtggagttctttgatgagaaacttaactccttgtgcatggcctggcttgtggatcatgtatatgccatccgcgaggcagccaccagcaacctgaagaagctagtggaaaagtttgggaaggagtgggcccatgccacaatcatccccaaggtcttggccatgtccggagaccccaactacctgcaccgcatgactacgctcttctgcatcaatgtgctgtctgaggtctgtgggcaggacatcaccaccaagcacatgctacccacggttctgcgcatggctggggacccggttgccaatgtccgcttcaatgtggccaagtctctgcagaagatagggcccatcctggacaacagcaccttgcagagtgaagtcaagcccatcctagagaagctgacccaggaccaggatgtggacgtcaaatactttgcccaggaggctctgactgttctgtctctcgcctgatgctggaagaggagcaaacactggcctctggtgtccaccctccaacccccacaagtccctctttggggagacactggggggcctttggctgtcactccctgtgcatggtctgaccccaggccccttcccccagcacggttcctcctctccccagcctgggaagatgtctcactgtccacctcccaacggctaggggagcacggggttggacaggacagtgaccttgggaggaaggggctactccgccatccttaaaagccatggagccggaggtggcaattcaccgaattc

SEQ ID NO: 7 - protein, 589 aa, Homo sapiens
MAAADGDDSLYPIAVLIDELRNEDVQLRLNSIKKLSTIALALGVERTRSELLPFLTDTIYDEDEVLLALAEQLGTFTTLVGGPEYVHCLLPPLESLATVEETVVRDKAVESLRAISHEHSPSDLEAHFVPLVKRLAGGDWFTSRTSACGLFSVCYPRVSSAVKAELRQYFRNLCSDDTPMVRRAAASKLGEFAKVLELDNVKSEIIPMFSNLASDEQDSVRLLAVEACVNIAQLLPQEDLEALVMPTLRQAAEDKSWAVRYMVADKFTELQKAVGPEITKTDLVPAFQNLMKDCEAEVRAAASHKVKEFCENLSADCRENVIMSQILPCIKELVSDANQHVKSALASVIMGLSPILGKDNTIEHLLPLFLAQLKDECPEVRLNIISNLDCVNEVIGIRQLSQSLLPAIVELAEDAKWRVRLAIIEYMPLLAGQLGVEFFDEKLNSLCMAWLVDHVYAIREAATSNLKKLVEKFGKEWAHATIIPKVLAMSGDPNYLHRMTTLFCINVLSEVCGQDITTKHMLPTVLRMAGDPVANVRFNVAKSLQKIGPILDNSTLQSEVKPILEKLTQDQDVDVKYFAQEALTVLSLA

SEQ ID NO: 8 - DNA, 1770 bp, Mus musculus
atggcagctgccgacggtgacgattcgctctatcctattgcggtgctcatagatgaactccgcaatgaggacgttcagcttcgtctcaatagtatcaagaagctctccacaattgccttggcccttggggttgaacggaccagaagtgagctcctgcccttccttacagataccatttatgatgaagatgaggtcctcttggccttggctgaacagctgggaaccttcacaactttggtgggagggcctgagtatgtgcactgtctgctgccaccccttgagtcactggccacagtggaagagacagtagtgcgagacaaggcggtagaatccttgcgggccatctctcatgagcactcaccttccgatctagaggctcactttgtgcctctggtaaagcggctggcgggtggagactggttcacctcccgcacctcggcctgtggtctcttctcagtttgctacccccgagtatccagtgccgtgaaggcagaacttcgacagtacttccggaacctgtgctcagatgacacccccatggtgcggcgggccgctgcctccaagctgggggaatttgccaaggtactggagctggacaatgtcaagagtgagatcattcccatgttctctaacctggcctctgacgagcaggactcggtgcggctgctggcagtggaggcatgtgtgaatattgcccagcttctgccacaggaggacctggaggccttagtgatgcccaccttgcgacaggctgctgaggacaagtcttggcgtgttcgctacatggtggccgacaagttcacagagctccagaaagcagtggggcctgagatcaccaagacagatctggtgcctgccttccagaacctgatgaaggactgtgaggccgaggtgagggccgcagcctcccacaaggtcaaagagttctgtgaaaatctctcagctgactgccgggagaatgtgatcatgactcagatcttgccctgcatcaaggagcttgtgtcagatgccaaccaacatgtcaagtcagcactggcttcagtcatcatgggcctctctcccattctgggcaaagacaacaccatcgaacacctcttgcccctgttcttggctcagctgaaggatgagtgtcctgaagtccgactgaatatcatctccaacctggattgtgtgaacgaggtgattggcatcaggcagctctctcagtccctgcttcctgccatcgtggaactagctgaagatgccaagtggcgagtgcggctggccatcattgaatacatgcctctgctggctggacagcttggtgtggaattttttgatgagaaactaaactctttgtgtatggcctggctagtggatcatgtctatgctatccgtgaggctgccaccagcaaccttaagaaattagtagagaagttcgggaaggagtgggcccatgccactatcatccccaaggttttagccatgtctggagaccctaactacctgcaccgaatgactacactcttctgcatcaatgtgttgtctgaggtctgtggacaggatatcaccaccaagcacatgctgcccacagttcttcgtatggcaggggacccagttgccaatgtccgcttcaatgtggccaagtcactccagaagataggacccattcttgataacagcaccctgcagagtgaagtcaagcccatcctggagaagctgacccaggaccaggatgtggatgtcaagtactttgcccaggaggctctgactgttctctctcttgcctga

SEQ ID NO: 9 - protein, 589 aa, Mus musculus
MAAADGDDSLYPIAVLIDELRNEDVQLRLNSIKKLSTIALALGVERTRSELLPFLTDTIYDEDEVLLALAEQLGTFTTLVGGPEYVHCLLPPLESLATVEETVVRDKAVESLRAISHEHSPSDLEAHFVPLVKRLAGGDWFTSRTSACGLFSVCYPRVSSAVKAELRQYFRNLCSDDTPMVRRAAASKLGEFAKVLELDNVKSEIIPMFSNLASDEQDSVRLLAVEACVNIAQLLPQEDLEALVMPTLRQAAEDKSWRVRYMVADKFTELQKAVGPEITKTDLVPAFQNLMKDCEAEVRAAASHKVKEFCENLSADCRENVIMTQILPCIKELVSDANQHVKSALASVIMGLSPILGKDNTIEHLLPLFLAQLKDECPEVRLNIISNLDCVNEVIGIRQLSQSLLPAIVELAEDAKWRVRLAIIEYMPLLAGQLGVEFFDEKLNSLCMAWLVDHVYAIREAATSNLKKLVEKFGKEWAHATIIPKVLAMSGDPNYLHRMTTLFCINVLSEVCGQDITTKHMLPTVLRMAGDPVANVRFNVAKSLQKIGPILDNSTLQSEVKPILEKLTQDQDVDVKYFAQEALTVLSLA

SEQ ID NO: 10 - protein, 6 aa, Simian Virus 40.
HENRKL

SEQ ID NO: 11 - protein, 9 aa, Xenopus laevis
MKVLKKAMI

SEQ ID NO: 12 - protein, 9 aa, Rattus norvegicus
LKVLKKTVD

SEQ ID NO: 13 - protein, 9 aa, Homo sapiens
DKTNKKKEK

SEQ ID NO: 14 - protein, 9 aa, Mus musculus
EKGRKKDTA

SEQ ID NO: 15 - protein, 9 aa, Mus musculus
TKAVKKKEK

SEQ ID NO: 16 - protein, 9 aa, Mus musculus
TKPTKKKKV

SEQ ID NO: 17 - protein, 9 aa, Mus musculus
FRHLKKTSK

SEQ ID NO: 18 - protein, 9 aa, Mus musculus
EHKGKKARL

SEQ ID NO: 19 - protein, 9 aa, Simian virus 40
KHENRKLYR

SEQ ID NO: 20 - protein, 9 aa, Mus musculus
DHEHRKLRL

SEQ ID NO: 21 - protein, 9 aa, Homo sapiens
SKLSHKHLV

SEQ ID NO: 22 - protein, 9 aa, Homo sapiens
NKNFHKSTG

SEQ ID NO: 23 - protein, 9 aa, Polyomavirus small
HRELKDKCD

SEQ ID NO: 24 - protein, 9 aa, Polyomavirus medium
HRELKDKCD

SEQ ID NO: 25 - protein, 9 aa, Homo sapiens
PGPTRKHYQ